(19) United States
(12) Patent Application Publication
(10) Pub. No.: US 2008/0146614 A1
(43) Pub. Date: Jun. 19, 2008

(54) Therapeutic Agents

(76) Inventor: Leifeng Cheng, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 11/793,335

(22) PCT Filed: Dec. 21, 2005
(86) PCT No.: PCT/GB05/04977

§ 371 Date: Jun. 19, 2007

(30) Foreign Application Priority Data
Dec. 23, 2004 (GB) 0428075.6
Jun. 29, 2005 (GB) 0513208.9

(51) Int. Cl. A61K 31/445; A61K 31/415; C07D 401/02; C07D 231/10; A61P 25/00; A61P 3/00; A61P 11/00; A61P 15/00; A61P 37/00; A61P 9/00

(52) U.S. Cl. 514/326; 514/406; 546/211; 548/377.1

(57) ABSTRACT

The present invention relates to 1,5-diphenylpyrazole compounds of formula I (A chemical formula should be inserted here—please see paper copy enclosed herewith) and processes for preparing such compounds, their use in the treatment of obesity, psychiatric and neurological disorders, to methods for their therapeutic use and to pharmaceutical compositions containing them.

### FIELD OF INVENTION

The present invention relates to certain 1,5-diphenylpyrazole compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties. Pyrazoles having anti-inflammatory activity are disclosed in WO99/64415 and EP 418 845. 1,5-Diarylpyrazole-3-carboxamide derivatives are disclosed as having CB₁ modulatory activity in U.S. Pat. No. 5,624,941, WO03/020217 and EP 656354. 1,5-Diarylpyrazole-3-carboxamide derivatives having a 4-hydroxymethyl substituent are disclosed in US 2004/0192667, EP 0876350 and EP 1,571,147 as having CB₁ modulatory activity. Co-pending application WO2005/080343 discloses 4-[1-(substituted phenyl)-3-[(carboxamido]-1H-pyrazol-5-yl]phenyl 1-alkanesulfonic acid ester derivatives as having CB₁ modulatory activity.

### DESCRIPTION OF THE INVENTION

The invention relates to a compound of formula (I)

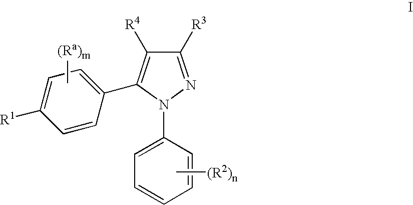

and pharmaceutically acceptable salts thereof, in which

R¹ represents a) a C₁₋₃alkoxy group optionally substituted by one or more of the following

i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group b) R¹ represents a C₄₋₆alkoxy group optionally substituted by one or more of the following i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group c) a group of formula phenyl(CH₂)ₚO— in which p is 1, 2 or 3 and the phenyl ring is optionally substituted by 1, 2 or 3 groups represented by Z, d) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z e) a group of formula (R⁶)₃ Si in which R⁶ represents a C₁₋₆alkyl group which may be the same or different or f) a group of formula R^{b}O(CO)O in which R^{b} represents a C₁₋₆alkyl group optionally substituted by one or more fluoro;

R^{a} represents halo, a C₁₋₃alkyl group or a C₁₋₃alkoxy group

m is 0, 1, 2 or 3;

R² represents a C₁₋₃alkyl group, a C₁₋₃alkoxy group, hydroxy, nitro, cyano or halo

n is 0, 1, 2 or 3;

R³ represents

a) a group X—Y—NR⁷R⁸

in which X is CO or SO₂,

Y is absent or represents NH optionally substituted by a C₁₋₃alkyl group; and R⁷ and R⁸ independently represent:

a C₁₋₆alkyl group optionally substituted by 1, 2, or 3 groups represented by W;

a C₃₋₁₅cycloalkyl group optionally substituted by 1, 2, or 3 groups represented by W;

an optionally substituted (C₃₋₁₅cycloalkyl)C₁₋₃alkylene group optionally substituted by 1, 2, or 3 groups represented by W;

a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;

a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;

a group —(CH₂)ₜHet in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo wherein the alkyl and alkoxy group are optionally independently substituted by one of more fluoro;

or R⁷ represents H and R⁸ is as defined above;

or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;

wherein the heterocyclic group is optionally substituted by one or more C₁₋₃alkyl groups, hydroxy, fluoro or benzyl;

or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;

R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;

Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and

W represents hydroxy, fluoro, a C₁₋₃alkyl group, a C₁₋₃alkoxy group, amino, mono or di C₁₋₃alkylamino, a C₁₋₆alkoxycarbonyl group or a heterocyclic amine selected from morpholinyl, pyrrolidinyl, piperidinyl or piperazinyl in which the heterocyclic amine is optionally substituted by a C₁₋₃alkyl group or hydroxyl.

In a particular group of compounds of formula I, R³ represents a group as described in paragraph a) above.

In a further particular group of compounds of formula I, R¹ represents a) a C₁₋₃alkoxy group substituted by one or more of the following i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group.

It will be understood that where a substituent Z is present in more than one group that these substituents are independently selected and may be the same or different. The same is true for W. Similarly when m is 2 or 3 then the groups R^{a} are independently selected so that they may be the same or different and similarly when n is 2 or 3 then the groups R² are independently selected so that they may be the same or different.

The term C₃₋₁₅cycloalkyl includes monocyclic, bicyclic, tricyclic and spiro systems for example, cyclopentyl, cyclohexyl and adamantyl.

The term heteroaryl means an aromatic 5-, 6-, or 7-membered monocyclic ring or a 9- or 10-membered bicyclic ring, with up to five ring heteroatoms selected from oxygen, nitrogen and sulfur. Suitable aromatic heteroaryl groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl. Preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl.

Suitable saturated or partially unsaturated 5 to 8 membered heterocyclic groups containing one or more heteroatoms selected from nitrogen, oxygen or sulphur include, for example tetrahydrofuranyl, tetrahydropyranyl, 2,3-dihydro-1,3-thiazolyl, 1,3-thiazolidinyl, pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyran-4-yl, pyrrolidin-3-yl, morpholino, piperidino, piperidin-4-yl or piperazin-1-yl.

A particular group of compounds of formula I is represented by formula IA

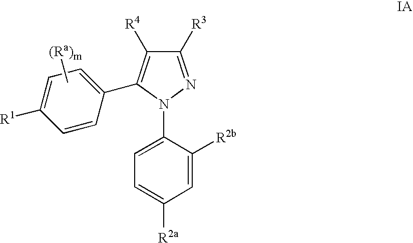

in which R¹ is
a) a C₄₋₆alkoxy group optionally substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z d) a group of formula (R⁶)₃ Si in which R⁶ represents a C₁₋₆alkyl group which may be the same or different or e) a group of formula R^{b}O(CO)O in which R^{b} represents a C₁₋₆alkyl group optionally substituted by one or more fluoro;

R^{a} represents halo and m is 0, 1 or 2;

R^{2a} represents H or chloro;

R^{2b} represents chloro;

R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by a C₁₋₆alkoxycarbonyl group or by one or more fluoro or hydroxy or R⁸ represents pyridyl optionally substituted by one or more W; and

R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

Particularly in compounds of formula IA, R³ represents a group CONHNR⁷R⁸ in which NR⁷R⁸ represents piperidino or morpholino or R³ represents a C₅₋₇cycloalkyl group optionally substituted by a C₁₋₆alkoxycarbonyl group. Particularly R^{2a} represents chloro. A further particular group of compounds of formula I is represented by formula IB

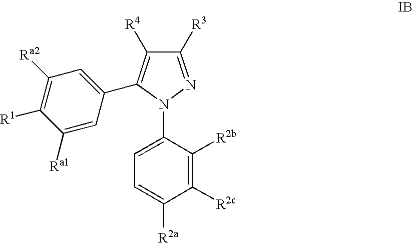

in which R¹ is
a) a C₄₋₆alkoxy group optionally substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;

R^{a1} represents halo or H;

R^{a2} represents halo or H;

R^{2a} represents H or chloro;

R^{2b} represents chloro;

R^{2c} represents halo or H;

R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by one or more fluoro or hydroxy or R⁸ represents pyridyl optionally substituted by trifluoromethyl; and

R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group. Particularly R^{2a} represents chloro.

In a particular group of compounds of formula IB, R³ represents a group CONHNR⁷R⁸ in which NR⁷R⁸ represents piperidino.

In one particular group of compounds of formula I, formula IA or formula IB, R¹ represents a C₄₋₆alkoxy group optionally substituted by one or more fluoro.

In a further particular group of compounds of formula I, formula IA or formula IB, R¹ represents a group R⁵S(O)₂O or R⁵S(O)₂NH in which R⁵ represents a C₁₋₆alkyl group optionally substituted by one or more fluoro, or R⁵ represents a heteroaryl group optionally substituted by 1, 2 or 3 groups represented by Z. In a yet further group of compounds of formula I, formula IA or formula IB, R¹ represents a group R⁵S(O)₂O in which R⁵ represents a C₁₋₆alkyl group optionally substituted by one or more fluoro. In a yet further group of compounds of formula I, formula IA or formula IB, R¹ represents a group R⁵S(O)₂O in which R⁵ represents a C₁₋₆alkyl group substituted by one or more fluoro.

In a still further particular group of compounds of formula I, formula IA or formula IB, R¹ represents a group of formula (R⁶)₃ Si in which R⁶ represents a C₁₋₆alkyl group which may be the same or different. In a still further particular group of compounds of formula I, formula IA or formula IB, R¹ is a group R⁵S(O)₂O in which R⁵ represents a C₃₋₆alkyl group substituted by one or more fluoro.

A particular group of compounds of formula I is represented by formula IC

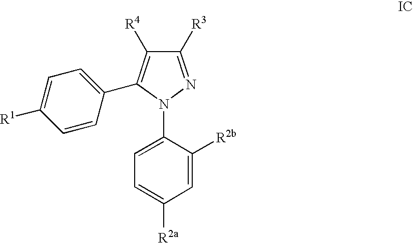

in which R¹ is
a) a C₄₋₆alkoxy group optionally substituted by one or more fluoro, b) a group R⁵S(O)₂O in which R⁵ represents a C₁₋₆alkyl group optionally substituted by one or more fluoro;

R^{2a} represents H or chloro;

R^{2b} represents chloro;

R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by a C₁₋₆alkoxycarbonyl group or by one or more fluoro or hydroxy or R⁸ represents pyridyl optionally substituted by trifluoromethyl; and

R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

In a particular group of compounds of formula IC, R^{2a} represents chloro.

In a particular group of compounds of formula IC, R³ represents a group CONHNR⁷R⁸ in which NR⁷R⁸ represents piperidino or morpholino.

In a particular group of compounds of formula IC, R¹ is a group R⁵S(O)₂O in which R⁵ represents a C₁₋₆alkyl group substituted by one or more fluoro. More particularly in compounds of formula IC, R¹ is a group R⁵S(O)₂O in which R⁵ represents a C₃₋₆alkyl group substituted by one or more fluoro.

Further values of R¹, R³ and R⁴ in compounds of formula I, formula IA, formula IB, or formula IC, now follow. It will be understood that such values may be used where appropriate with any of the definitions, claims or embodiments defined hereinbefore or hereinafter.

Particularly R¹ is a C₄₋₆alkoxy group substituted by one or more fluoro for example 3-fluoropropoxy, or 3,3,3,trifluoropropoxy. More particularly R¹ is 4,4,4-trifluorobutoxy, n-butylsulfonyloxy, n-propylsulfonyloxy, 4,4,4-trifluorobutyl-1-sulfonyloxy, 3,3,3-trifluoropropyl-1-sulfonyloxy or propoxycarbonyloxy. Most particularly R¹ is 3,3,3-trifluoropropyl-1-sulfonyloxy, or n-propylsulfonyloxy especially 3,3,3-trifluoropropyl-1-sulfonyloxy.

In a further particular group of compounds of formula I, formula IA, formula IB, or formula IC, m is O, R^{2a} is H or chloro and R^{2b} is chloro.

In a further particular group of compounds of formula I, formula IA, formula IB, or formula IC, R³ represents N-(piperidin-1-yl)carbamoyl, N-(4,4-difluorocyclohexyl)carbamoyl, N-(5-trifluoromethyl-2-pyridyl)carbamoyl, N-(cyclohexyl)carbamoyl or N-(2-hydroxycyclohexyl)carbamoyl. In a further particular group of compounds of formula I, formula IA, formula IB, or formula IC, R³ represents N-(piperidin-1-yl)carbamoyl.

In a particular group of compounds of formula I, formula IA, formula IB, or formula IC, R⁴ represents a group of formula CH₂NR^{e}R^{f} in which R^{e} and R^{f} are as previously defined.

In a further particular group of compounds of formula I, formula IA, formula IB, or formula IC, R⁴ represents a group of formula CH₂OH.

In a particular group of compounds of formula I, formula IA, formula IB, or formula IC, R⁴ represents a group of formula CH₂NH₂ or CH₂N(CH₃)₂.

“Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid or, for example a base-addition salt of a compound of Formula I which is sufficiently acidic for example a base-addition with an inorganic or an organic base.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisormers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl, t-butyl, pentyl, isopentyl, neopentyl, tert-pentyl, hexyl and isohexyl. Preferred alkyl groups are methyl, ethyl, propyl, isopropyl, butyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Specific compounds of the invention include one or more of the following:
propane-1-sulfonic acid 4-[2-(2,4-dichlorophenyl)-4-hydroxymethyl-5-(piperidin-1-ylcarbamoyl)-2H-pyrazol-3-yl]phenyl ester;
propane-1-sulfonic acid 4-[4-aminomethyl-2-(2,4-dichlorophenyl)-5-(piperidin-1-ylcarbamoyl)-2H-pyrazol-3-yl]phenyl ester;
1-(2,4-dichlorophenyl)-4-hydroxymethyl-5-(4-methoxyphenyl)-1H-pyrazole-3carboxylic acid (4,4-difluoro-cyclohexyl)amide;
3,3,3-trifluoropropane-1-sulfonic acid 4-[2-(2,4-dichlorophenyl)-5-(4,4-difluoro-cyclohexylcarbamoyl)-4-hydroxymethyl-2H-pyrazol-3-yl]phenyl ester;
3,3,3-trifluoro-propane-1-sulfonic acid 4-[2-(2,4-dichlorophenyl)-4-hydroxymethyl-5-(piperidin-1-ylcarbamoyl)-2H-pyrazol-3-yl]phenyl ester;
propane-1-sulfonic acid 4-[2-(2,4-dichlorophenyl)-5-(4,4-difluorocyclohexylcarbamoyl)-4-hydroxymethyl-2H-pyrazol-3-yl]phenyl ester;
3,3,3-trifluoropropane-1-sulfonic acid 4-[2-(2,4-dichlorophenyl)-4-hydroxymethyl-5-(5-trifluoromethylpyridin-2-ylcarbamoyl)-2H-pyrazol-3-yl]phenyl ester;
3,3,3-trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester;
3,3,3-trifluoropropane-1-sulfonic acid 4-[5-cyclohexylcarbamoyl-2-(2,4-dichloro-phenyl)-4-hydroxymethyl-2H-pyrazol-3-yl]phenyl ester;
N-cyclohexyl-1-(2,4-dichlorophenyl)-4-hydroxymethyl-5-[4-(3,3,3-trifluoropropoxy)phenyl]-1H-pyrazole-3-carboxamide;
1-(2,4-Dichlorophenyl)-N-(4,4-difluorocyclohexyl)-4-hydroxymethyl-5-[4-(3,3,3-trifluoropropoxy)phenyl]-1H-pyrazole-3-carboxamide;
N-cyclohexyl 1-(2,4-dichlorophenyl)-4-[(dimethylamino)methyl]-5-[4-(3-fluoropropoxy)phenyl]-1H-pyrazole-3-carboxamide;
N-cyclohexyl 1-(2,4-dichlorophenyl)-4-[(dimethylamino)methyl]-5-[4-(3-fluoropropoxy)phenyl]-1H-pyrazole-3-carboxamide;
3,3,3-Trifluoropropane-1-sulfonic acid 4-[5-cyclohexylcarbamoyl-2-(2,4-dichloro-phenyl)-4-dimethylaminomethyl-2H-pyrazol-3-yl]phenyl ester;
4-[1-(2-chlorophenyl)-3-[(cyclohexylamino)carbonyl]-4-(hydroxymethyl)-1H-pyrazol-5-yl]phenyl 3,3,3-trifluoropropane-1-sulfonate;
4-[1-(2-chlorophenyl)-3-({[(1S,2R)-2-hydroxycyclohexyl]amino}carbonyl)-4-(hydroxymethyl)-1H-pyrazol-5-yl]phenyl 3,3,3-trifluoropropane-1-sulfonate and 4-[1-(2-chlorophenyl)-3-({[(1R,2S)-2-hydroxycyclohexyl]amino}carbonyl)-4-(hydroxymethyl)-1H-pyrazol-5-yl]phenyl 3,3,3-trifluoropropane-1-sulfonate
   as well as pharmaceutically acceptable salts thereof.

### Methods of Preparation

Compounds of formula I in which R^{a}, R¹, R², R³, m and n are as previously defined and R⁴ represents CH₂NH₂ may be prepared by reacting a compound of formula II

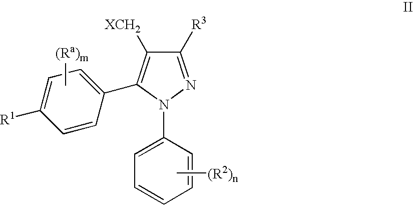

in which R^{a}, R¹, R², R³, m and n are as previously defined and X represents phthalimido with hydrazine hydrate in the presence of a solvent for example methanol at a temperature in the range of 15-150° C.

Compounds of formula I in which R^{a}, R¹, R², R³, m and n are as previously defined and R⁴ represents CH₂OH may be prepared by reacting a compound of formula II

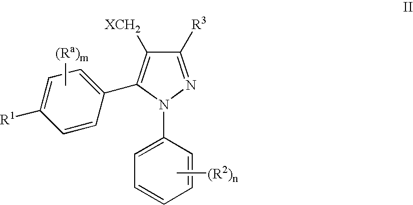

in which R^{a}, R¹, R², R³, m and n are as previously defined and X represents a leaving group for example halo e.g. bromo, chloro or iodo, with a hydrolysing agent for example silver nitrate in the presence of a solvent system for example aqueous acetone at a temperature in the range of 15-150° C.

Compounds of formula I in which R^{a}, R¹, R², R³, m and n are as previously defined and R⁴ represents CH₂NR^{e}R^{f} in which R^{e} and R^{f} are as previously defined may be prepared by reacting a compound of formula II

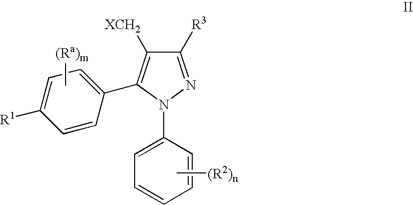

in which R^{a}, R¹, R², R³, m and n are as previously defined and X represents a leaving group for example halo e.g. bromo, chloro or iodo, with an amine of formula HNR^{e}R^{f} in which R^{e} and R^{f} are as previously defined in an inert solvent, for example ethanol, at a temperature in the range of 15-150° C.

Compounds of formula I in which R^{a}, R², R³, R⁴, m and n are as previously defined and R¹ represents a group R⁵S(O)₂NH may be prepared by reacting a compound of formula III

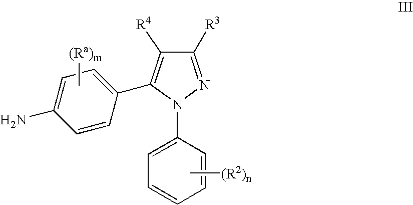

in which R^{a}, R², R³, R⁴, m and n are as previously defined with a sulphonating agent of formula R⁵SO₂L in which R⁵ is as previously defined and L represents a leaving group, for example chloro, in an inert solvent, for example dichloromethane, in the presence of a base, for example triethylamine, at a temperature in the range of −25° C. to 150° C.

Compounds of formula I in which R¹ represents a) a C₁₋₃alkoxy group substituted by one or more fluoro or C₄₋₆alkoxy group optionally substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O may be prepared by reacting a compound of formula IV

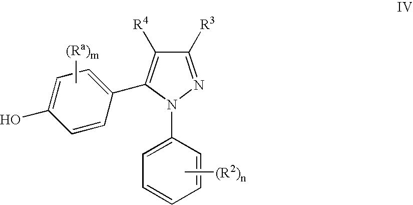

in which R^{a}, R², R³, R⁴, m and n are as previously defined with either
a) an alkylating agent of formula R⁹X in which R⁹ represents a C₁₋₃alkyl group substituted by one or more fluoro or C₄₋₆alkyl group optionally substituted by one or more fluoro and X represents a leaving group, for example chloro, bromo, iodo, mesylate or triflate in an inert solvent, for example acetone, in the presence of a base, for example potassium carbonate, at a temperature in the range of −25° C. to 150° C.; or
b) an alkylating agent of formula R⁹X in which R⁹ represents a group of formula phenyl(CH₂)ₚ— in which p is 1, 2 or 3 and the phenyl ring is optionally substituted by 1, 2 or 3 groups represented by Z, and X represents a leaving group, for example chloro, bromo or iodo, in an inert solvent, for example acetone, in the presence of a base, for example potassium carbonate, at a temperature in the range of −25° C. to 150° C.; or
c) a sulphonating agent of formula R⁵SO₂L in which R⁵ is as previously defined and L represents a leaving group, for example chloro, in an inert solvent, for example dichloromethane, in the presence of a base, for example triethylamine or pyridine, at a temperature in the range of −25° C. to 150° C.; respectively.

Compounds of formula I in which R^{a}, R¹, R², R⁴, m and n are as previously defined and R³ represents a group X—Y—NR⁷R⁸ in which X is CO, Y is absent or represents NH optionally substituted by a C₁₋₃alkyl group and R⁷ and R⁸ are as previously defined may also be prepared by reacting a compound of formula V

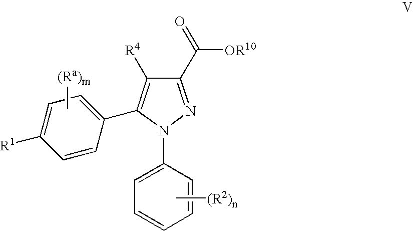

in which R^{a}, R¹, R², R⁴, m and n are as previously defined and R¹⁰ represents a C₁₋₆alkyl group with a compound of formula VI

R⁷R⁸YNH₂ VI

in which Y, R⁷ and R⁸ are as previously defined or a salt thereof in an inert solvent, for example toluene, in the presence of a Lewis acid, for example trimethylaluminum, at a temperature in the range of −25° C. to 150° C.

Compounds of formula I in which R³ represents a group X—Y—NR⁷R⁸ in which X is SO₂, Y is absent or represents NH optionally substituted by a C₁₋₃alkyl group and R⁷ and R⁸ are as previously defined may also be prepared by reacting a compound of formula VII

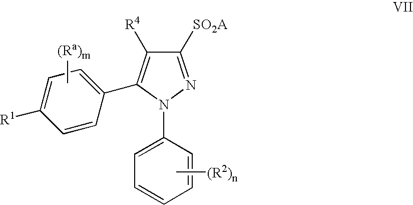

in which R^{a}, R¹, R², R⁴, m and n are as previously defined and A represents a leaving group, for example halo e.g. chloro, with a compound of formula VI in which Y, R⁷ and R⁸ are as previously defined or a salt thereof in an inert solvent, for example THF or dichloromethane in the presence of a base, for example potassium carbonate, triethylamine or pyridine, at a temperature in the range of −25° C. to 150° C.

Compounds of formula IV and V may be prepared by methods analogous to the following method.

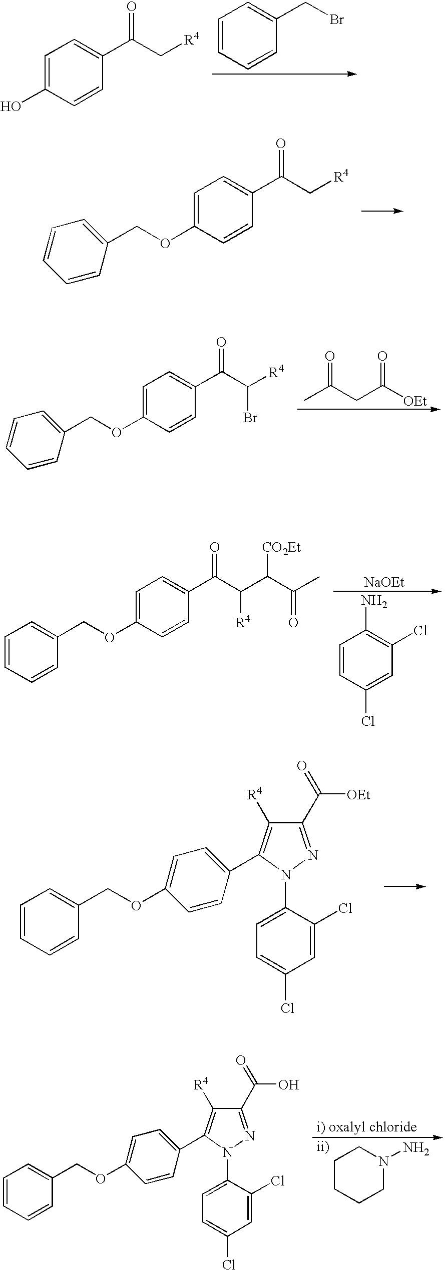

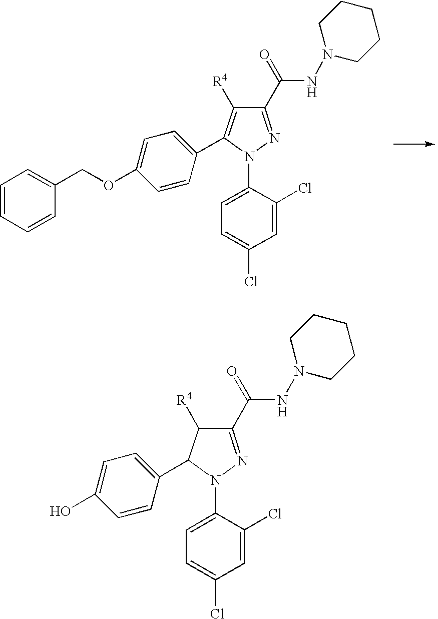

Certain intermediate compounds of formula II, III, IV, V and VII are believed to be novel and form part of the present invention. It will be appreciated by those skilled in the art that during the reaction sequence certain functional groups, for example hydroxy groups and optionally substituted amino groups in R⁴, will require protection followed by deprotection at an appropriate stage see “Protective Groups in Organic Synthesis”, 3rd Edition (1999) by Greene and Wuts.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse. In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

The compounds of the present invention may also be suitable for use in the regulation of bone mass and bone loss and therefore useful in the treatment of osteoporosis and other bone diseases.

The compounds of the present invention may also be used in the treatment of hepatic diseases, for example hepatic fibrosis, alcoholic liver cirrhosis, chronic viral hepatitis, non-alcoholic steatohepatitis or liver cancer.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

As the compounds of formula I are useful in causing smoking cessation, preventing weight gain resulting from smoking cessation, treating nicotine withdrawal and preventing nicotine dependence they may also be combined with other compounds known to have one or more of these effects for example nicotine, a nicotine agonist or a partial agonist, a monoamine oxidase inhibitor or antidepressants such as bupropion, doxepine, nortriptyline or an anxiolytic such as buspirone or clonidine.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar. For example, Example 1 has an IC50 of 3.3 nM

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES

### Abbreviations

AcOH acetic acid
AIBN 2,2′-azobisisobutyronitrile
BOP benzotriazol-1-yloxytris(dimethylamino)phosphonium hexafluorophosphate
DCM dichloromethane
DMF dimethylformamide
DEA diethylamine
DEAD diethyl azodicarboxylate

### DIEA N,N-diisopropylethylamine

DMAP 4-dimethylaminopyridine

### DMF N,N-dimethylformamide

EtOAc ethyl acetate
LiHMDS lithium hexamethyldisilazide
MeOH methanol
rt or RT room temperature
TEA triethylamine
THF tetrahydrofuran
TLC thin layer chromatography
t triplet
singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC (High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5).

For isolation of isomers, a Kromasil CN E9344 (250×20 mm i.d.) column was used.

Heptane:ethyl acetate:DEA 95:5:0.1 was used as mobile phase (1 ml/min). Fraction collection was guided using a UV-detector (330 nm).

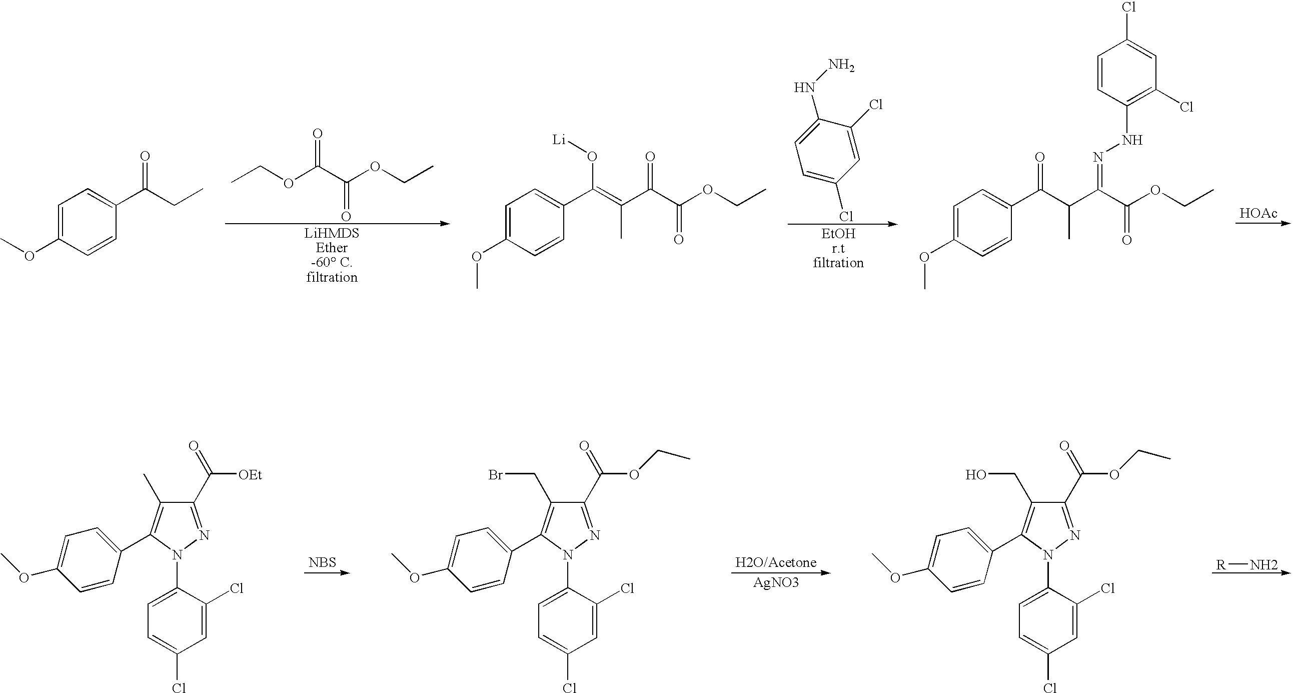

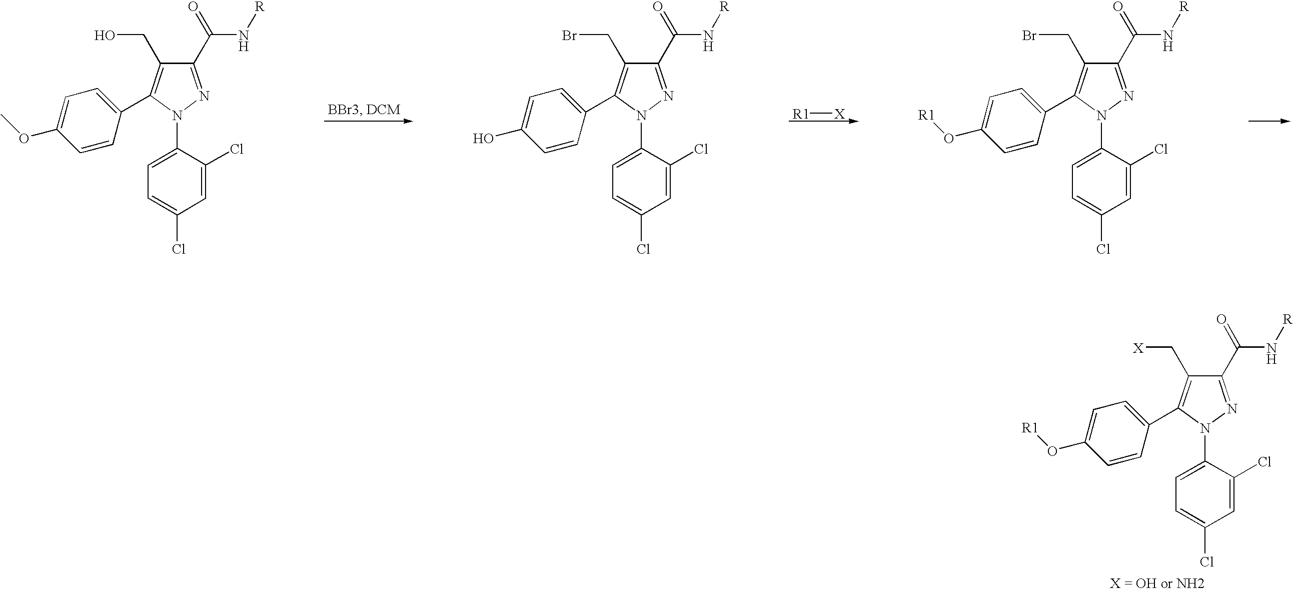

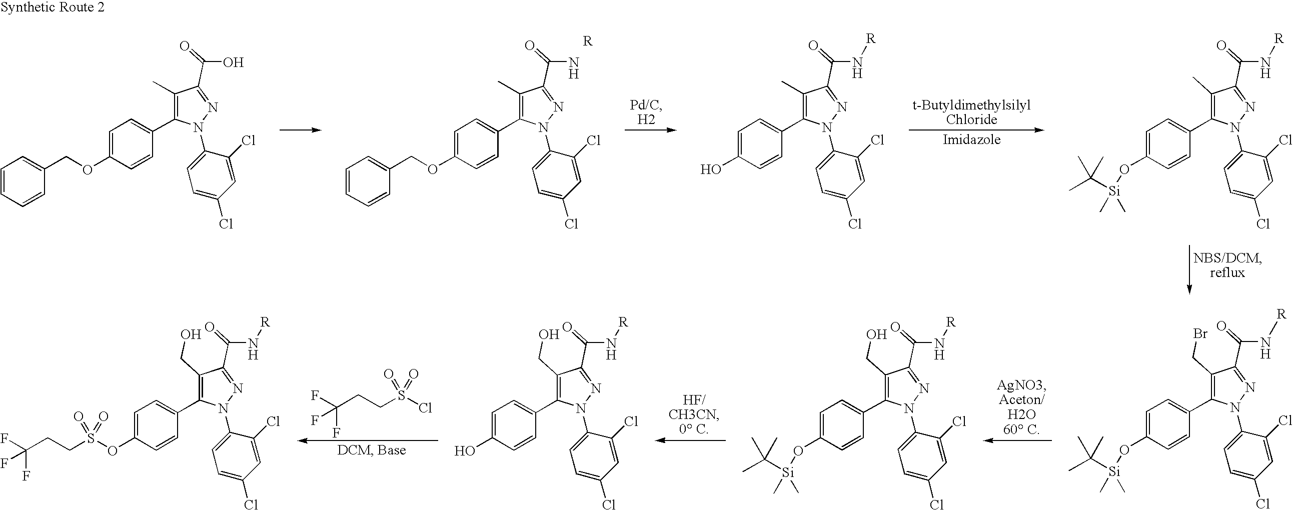

### Example 1

### Propane-1-sulfonic acid 4-[2-(2,4-dichlorophenyl-4-hydroxymethyl-5-(piperidin-1-ylcarbamoyl)-2H-pyrazol-3-yl]phenyl ester

### Step A: 5-(4-Methoxyphenyl)-1-(2,4-dichlorophenyl)-4-methyl-1H-pyrazole-3-carboxylic acid ethyl ester

To a magnetically stirred solution of lithium bis(trimethylsilyl)amide (generated from 1,1,1,3,3,3-hexamethyldisilazane, 16.1 g, 0.1 mol and butyllitium (67 ml, 1.6 M) 0.1 mol)) in heptane:ether (100:300 ml) was added 4-methoxypropiophenone (16.4 g, 0.10 mol) in ether (100 ml) at −78° C. After stirring at this temperature for further 45 min. diethyl oxalate (17.1 g, 0.12 mol) was added and the reaction mixture stirred at room temperature (rt) overnight. The precipitated material was filtered, washed with ether and dried to afford 15.5 g (64%) of the Lithium salt of ethyl 2,4-dioxo-3-methyl-4-(4-methoxyphenyl)butanoate as a pale yellow solid. To a magnetically stirred solution of lithium salt of ethyl 2,4-dioxo-3-methyl-4-(4-methoxyphenyl)butanoate (13.13 g, 48.6 mmol) in 200 ml ethanol was added 2,4-dichlorophenylhydrazine hydrochloride (13.13 g, 60.0 mmol) and the resulting mixture stirred at room temperature overnight. The precipitate was filtered and dried and the yellow solid suspended in acetic acid and heated at reflux overnight. After cooling to rt, water was added and the product extracted with EtOAc (×3). The combined organic extract was washed with brine, dried (Na₂SO₄), filtered and concentrated. Flash chromatography (Heptane:EtOAc gradient) afforded 6.50 g (33%) of the title compound.

### Step B: 4-Bromomethyl-1-(2,4-dichlorophenyl)-5-(4-methoxyphenyl-1H-pyrazole-3-carboxylic acid ethyl ester

To a magnetically stirred solution of 1-(2,4-dichlorophenyl)-5-(4-methoxyphenyl)-4-methyl-1H-pyrazole-3-carboxylic acid ethyl ester (2.97 g, 7.32 mmol) in carbon tetrachloride was added N-bromosuccinimde (1.44 g, 8.09 mmol) and 2,2′-azoisobutyronitrile (148 mg). The resulting mixture was refluxed for 2 hrs, cooled to rt and filtered. The solvent was removed under reduced pressure to give 3.54 g (100%) of the title compound as a pale yellow solid.

### Step C: 1-(2,4-Dichlorophenyl)-4-hydroxymethyl-5-(4-methoxyphenyl-1H-pyrazole-3-carboxylic acid ethyl ester

To a magnetically stirred solution of 4-bromomethyl-1-(2,4-dichlorophenyl)-5-(4-methoxyphenyl)-1H-pyrazole-3-carboxylic acid ethyl ester (3.30 g, 6.81 mmol) in 50% aqueous acetone (140 ml) was added silver nitrate (4.07 g, 23.8 mmol). The reaction mixture was stirred at 60° C. for 1.5 hrs, cooled to rt, water added and the product extracted with DCM (×3). The combined organic extracts were dried (Na₂SO₄), filtered and concentrated. Flash chromatography (Hexane:EtOAc gradient) afforded 2.59 (90%) of the title compound as a colorless solid.

### Step D: 1-(2,4-Dichlorophenyl)-4-hydroxymethyl-5-(4-methoxyphenyl)-1H-pyrazole-3-carboxylic acid piperidin-1-ylamide

To a magnetically stirred suspension of aluminium chloride (1.64 g, 12.3 mmol) in 1,2-dichloroethane (25 ml) was added 1-aminopiperidine (2.66 ml, 24.6 mmol) at 0° C. The suspension was allowed to warm to room temperature and a solution of 1-(2,4-dichloro-phenyl)-4-hydroxymethyl-5-(4-methoxyphenyl)-1H-pyrazole-3-carboxylic acid ethyl ester (2.59 g, 6.15 mmol) in 1,2-dichloroethane (25 ml) was added. The reaction mixture was stirred at room temperature overnight, then water was added carefully and the product extracted with DCM (×3). The combined organic extracts were dried (Na₂SO₄), filtered and concentrated. Flash chromatography (Hexane:EtOAc 50:50-EtOAc) afforded 2.17 g (74%) of the title compound as a colorless solid.

### Step E: 4-Bromomethyl-1-(2,4-dichlorophenyl)-5-(4-hydroxyphenyl-5-1H-pyrazole-3-carboxylic acid piperidin-1-ylamide

To a solution of 1-(2,4-dichlorophenyl)-4-hydroxymethyl-5-(4-methoxyphenyl)-1H-pyrazole-3-carboxylic acid piperidin-1-ylamide (1.44 g, 3.02 mmol) in dichloromethane (50 ml) at 0° C. was added boron tribromide (1.2 ml, 12.4 mmol). The cooling bath was removed and stirring continued for 2 hrs at room temperature before pouring it onto ice-water and extracting with DCM (×3). The combined extracts were dried (Na₂SO₄), filtered and concentrated. Flash chromatography (EtOAc) afforded 600 mg (38%) of the title compound as a colorless solid.

### Step F: Propane-1-sulfonic acid 4-[4-bromomethyl-2-(2,4-dichloro-phenyl)-5-(piperidin-1-ylcarbamoyl)-2H-pyrazol-3-yl]-phenyl ester

To a solution of 4-bromomethyl-1-(2,4-dichlorophenyl)-5-(4-hydroxyphenyl)-5-1H-pyrazole-3-carboxylic acid piperidin-1-ylamide (600 mg, 1.14 mmol) in dichloromethane (25 ml) was added triethylamine (0.32 ml, 2.28 mmol) and the reaction mixture cooled to 0° C. 1-Propanesulfonylchloride (0.33 g, 2.28 mmol) was added, the cooling bath removed and the reaction mixture stirred at rt for 2 hrs. Water was added, the product extracted with DCM (×2), and the combined organic extracts washed with water, dried (Na₂SO₄), filtered and concentrated. Flash chromatography (hexane:EtOAc gradient) afforded 490 mg (68%) of the title compound as a colorless solid.

### Step G: Propane-1-sulfonic acid 4-[2-(2,4-dichlorophenyl)-4-hydroxymethyl-5-(piperidin-1-ylcarbamoyl)-2H-pyrazol-3-yl]-phenyl ester

To a magnetically stirred solution of propane-1-sulfonic acid 4-[4-bromomethyl-2-(2,4-dichloro-phenyl)-5-(piperidin-1-ylcarbamoyl)-2H-pyrazol-3-yl]-phenyl ester (240 mg, 0.38 mmol) in 50% aqueous acetone (10 ml) was added silver nitrate (226 mg, 1.33 mmol). The reaction mixture was stirred at 60° C. for 1.5 hrs, cooled to rt, water added and the product extracted with DCM (×3). The combined organic extracts were dried (Na₂SO₄), filtered and concentrated. Flash chromatography (Hexane:EtOAc 50:50-EtOAc) afforded 120 mg (56%) of the title compound as a colorless solid.

¹H NMR (CDCl₃): [symbol] 7.40-7.20 (8H, m), 5.00 (1H, broad s), 4.60 (2H, m), 3.30-3.20 (2H, m), 3.10-2.90 (4H, m), 2.10-1.85 (6H, s), 1.60-1.40 (2H, m), 1.08 (3H, t). MS: 589 (M+Na)

### Example 2

### Propane-1-sulfonic acid 4-[4-aminomethyl-2-(2,4-dichlorophenyl)-5-(piperidin-1-ylcarbamoyl)-2H-pyrazol-3-yl]-phenyl ester

### Step A: Propane-1-sulfonic acid 4-[2-(2,4-dichlorophenyl)-4-(1,3-dioxo-1,3-dihydro-isoindol-2-ylmethyl)-5-(piperidin-1-ylcarbamoyl)-2H-pyrazol-3-yl]phenyl ester

To a solution of propane-1-sulfonic acid 4-[4-bromomethyl-2-(2,4-dichlorophenyl)-5-(piperidin-1-ylcarbamoyl)-2H-pyrazol-3-yl]-phenyl ester from Ex 1, Step F (250 mg, 0.44 mmol) in DMF (5 ml) was added potassium phthalimide (163 mg, 0.88 mmol). The reaction mixture was stirred at rt overnight, water added and the product extracted with EtOAc (×3). The combined organic extracts were washed with water, dried (Na₂SO₄), filtered and concentrated. Flash chromatography (hexane:EtOAc 50:50-EtOAc) afforded 150 mg (50%) of the title compound as a colorless solid.

### Step B: Propane-1-sulfonic acid 4-[4-aminomethyl-2-(2,4-dichlorophenyl)-5-(piperidin-1-ylcarbamoyl)-2H-pyrazol-3-yl]-phenyl ester

To a solution of propane-1-sulfonic acid 4-[2-(2,4-dichlorophenyl)-4-(1,3-dioxo-1,3-dihydro-isoindol-2-ylmethyl)-5-(piperidin-1-ylcarbamoyl)-2H-pyrazol-3-yl]-phenyl ester (150 mg, 0.21 mmol) in methanol (5 ml) was added 0.1 ml hydrazine hydrate and the reaction mixture refluxed for 1 hour. Evaporation and flash chromatography (EtOAc:MeOH 80:20) gave a crude product that was suspended in EtOAc, the precipitated material filtered off and the filtrate concentrated to give 65 mg (55%) of the title compound as a colorless solid.

MS: 566 (M+Na). HPLC: 93.4%

### Example 3

### 1-(2,4-Dichlorophenyl)-4-hydroxymethyl-5-(4-methoxyphenyl)-1H-pyrazole-3carboxylic acid (4,4-difluoro-cyclohexyl)amide

### Step A 1-(2,4-Dichlorophenyl)-5-(4-methoxyphenyl)-1-methyl-1H-pyrazole-3-carboxylic acid ethyl ester

To a magnetically stirred solution of lithium bis(trimethylsilyl)amide (200 ml, 1 M solution in hexane, 0.2 mol) in ether (600 ml) was added a solution of 4-methoxypropiophenone (32.84 g, 0.20 mol) in ether (200 ml) at −78° C. After stirring at this temperature for further 45 mins diethyl oxalate (34.5 g, 0.235 mol) was added and the reaction mixture stirred at room temperature overnight. The precipitated material was collected by filtration, washed with ether and dried to afford 30.81 g (57%) of the lithium salt as a pale yellow solid. To a magnetically stirred solution of this lithium salt (30.81, 0.11 mol) in 450 ml ethanol was added 2,4-dichlorophenylhydrazine hydrochloride (46.9 g, 0.22 mol) and the resulting mixture stirred at room temperature overnight. The precipitated material was filtered off, dried, dissolved in acetic acid and boiled under reflux overnight. Ice-water was added and the product extracted with EtOAc (×3). The combined organic extracts were washed with water, dried (Na₂SO₄), filtered and concentrated. Flash chromatography (Heptane:EtOAc gradient) afforded 19.4 g (24%) of the title compound.

### Step B 4-Bromomethyl-1-(2,4-dichlorophenyl)-5-(4-methoxyphenyl)-1H-pyrazole-3-carboxylic acid ethyl ester

To a magnetically stirred solution of 1-(2,4-dichlorophenyl)-4-methyl-5-(4-methoxyphenyl)-1H-pyrazole-3-carboxylic acid ethyl ester (6.64 g, 16.4 mmol) in 1,2-dichloroethane (280 ml) was added N-bromosuccinimide (3.18 g, 17.8 mmol) and 2,2-azoisobutyronitrile (355 mg, 2.16 mmol). The resulting mixture was boiled under reflux for 2 hours, concentrated and purified by flash chromatography (heptane:EtOAc gradient) to give 8.05 g of the title compound used directly in the next step.

### Step C 1-(2,4-Dichlorophenyl)-4-hydroxymethyl-5-(4-methoxyphenyl)-1H-pyrazole-3-carboxylic acid

To a solution of 4-bromomethyl-1-(2,4-dichlorophenyl)-5-(4-methoxyphenyl)-1H-pyrazole-3-carboxylic acid ethyl ester (8.00 g, 16.5 mmol) in THF (60 ml) was added 5% NaOH (60 ml) and the reaction mixture boiled under reflux for 3 hours. After cooling to room temperature, the reaction mixture was acidified to pH 3 with HCl and the product extracted with dichloromethane (×3). The combined organic extracts were washed with water, dried (Na₂SO₄), filtered and concentrated to give 6.31 g (97%) of the product as a pale yellow solid.

### Step D 1-(2,4-Dichlorophenyl)-4-hydroxymethyl-5-(4-methoxyphenyl)-1H-pyrazole-3-carboxylic acid (4,4-difluoro-cyclohexyl)-amide

To a solution of 1-(2,4-dichlorophenyl)-4-hydroxymethyl-5-(4-methoxyphenyl)-1H-pyrazole-3-carboxylic acid (2.08 g, 5.30 mmol) in dichloromethane was added triethylamine (1.88 ml, 13.4 mmol) and 4,4-difluorocyclohexylamine (0.76 g, 5.60 mmol). The reaction mixture was cooled to 0° C. and BOP added. The reaction mixture was stirred at rt overnight, poured into ice-water, extracted with DCM (×3), washed with water, dried (Na₂SO₄), filtered and concentrated. Flash chromatography (Heptane:EtOAc) afforded 1.40 g (52%) of the title compound as a colorless solid.

¹H NMR (CDCl₃): [symbol] 7.40-7.20 (3H, m), 7.15-7.00 (2H, m), 6.95-6.80 (2H, m), 4.65 (2H, s), 4.20-4.00 (1H, broad s), 3.80 (3H, s), 2.30-1.60 (8H, m).

HPLC: 98.5%. MS: 492 (M−H₂O), 510 (M+H)

### Example 4

### 3,3,3-Trifluoropropane-1-sulfonic acid 4-[2-(2,4-dichlorophenyl)-5-(4,4-difluoro-cyclohexylcarbamoyl)-4-hydroxymethyl-2H-pyrazol-3-yl]phenyl ester

### Step A 1-(4-Benzyloxyphenyl)propan-1-one

4-Hydroxypropiophenone (15.0 g, 0.10 mol) was dissolved in acetone (200 ml) together with potassium carbonate (13.8 g, 0.10 mol). Benzyl bromide (17.1 g, 0.10 mol) was added and the reaction mixture heated at reflux overnight. After cooling to room temperature the mixture was filtered and concentrated on the rotary evaporator to afford 24.0 g (100%) of the title compound as a white solid

### Step B 1-(4-Benzyloxyphenyl)-2-bromopropan-1-one

1-(4-Benzyloxyphenyl)propan-1-one (4.80 g, 20.0 mmol) was suspended in acetic acid (25 ml) and cooled to 0° C. Bromine (3.20 g, 20.0 mmol) was added dropwise and the reaction mixture stirred two hours at room temperature at which point the reaction mixture was a clear, yellow solution. After cooling, water (100 ml) was added and the product extracted with ether (2×100 ml). The combined organic extracts were washed with water, sodium hydrogen carbonate and brine. The organic phase was dried (Na₂SO₄), filtered and evaporated leaving the title compound as a pale yellow solid (6.17 g, 97%).

### Step C 2-[2-(4-Benzyloxy-phenyl)-2-oxo-ethyl]-3-oxo-butyric acid ethyl ester

A solution of sodium ethoxide was generated from sodium metal (0.53 g, 23.0 mmol) in 30 ml abs. ethanol. To this solution was added ethyl acetoacetate (3.00 g, 23.0 mmol) at 0° C. After 30 min. this solution was added to a solution of 1-(4-benzyloxyphenyl)-2-bromo-propan-1-one (6.17 g, 19.0 mmol) in ethanol:toluene (30:15 ml) and the reaction mixture stirred overnight. Acidic work-up with 1 M HCl, extraction with ethyl acetate (3×), washing with brine, drying (Na₂SO₄), filtering and evaporation left a crude product purified by flash chromatography (hexane:EtOAc 95:5-70:30) affording 5.18 g of the title compound as a pale yellow oil.

### Step D 5-(4-Benzyloxyphenyl)-1-(2,4-dichlorophenyl)-4-methyl-1H-pyrazole-3-carboxylic acid

A solution of sodium ethoxide was generated from sodium metal (0.19 g, 8.26 mmol) in 20 ml abs. ethanol. To this solution was added 2-[2-(4-benzyloxyphenyl)-2-oxo-ethyl]-3-oxo-butyric acid ethyl ester (2.13 g, 6.00 mmol) and the reaction mixture stirred at room temperature for 30 min. A previously prepared solution of 2,4-dichlorophenyldiazonium chloride (prepared from 2,4-dichloroaniline (1.19 g, 7.30 mmol) in 3 ml 24% HCl and sodium nitrite (0.52 g, 7.50 mmol) in 3 ml water at 0° C.) was added in 5 portions keeping the temperature below 5° C. After stirring at room temperature for 2.5 hours water was added, and the product extracted with EtOAc (3×). The combined organic extracts were dried (Na₂SO₄), filtered and evaporated. The residue was dissolved in ethanol (40 ml) and sodium hydroxide (0.80 g, 20.0 mmol) in 10 ml of water was added. After 2 hours boiling under reflux the reaction mixture was cooled, acidified with HCl and the product extracted with EtOAc (3×). After washing, drying (Na₂SO₄), filtration and concentration, the residue was purified by flash chromatography (hexane:EtOAc 70:30-50:50) affording 1.84 g (68%) of the title compound as a pale yellow solid.

### Step E 5-(4-Benzyloxyphenyl)-1-(2,4-dichlorophenyl)-4-methyl-1H-pyrazole-3-carboxylic acid (4,4-difluorocyclohexyl)-amide

To a solution of 5-(4-benzyloxyphenyl)-1-(2,4-dichlorophenyl)-4-methyl-1H-pyrazole-3-carboxylic acid (1.00 g, 2.22 mmol) and 4,4-difluorocyclohexylamine (0.30 g, 2.22 mmol) was added triethylamine (0.79 ml, 5.62 mmol). The reaction mixture was cooled to 0° C. and BOP added with stirring. The reaction mixture was allowed to reach room temperature and stirred overnight. Water was added and the product extracted with DCM (×3). The combined organic extracts were washed with water, dried (Na₂SO₄), filtered and concentrated. Flash chromatography (heptane:EtOAc 70:30) gave 1.16 g (92%) of the title compound as a yellow solid.

### Step F 1-(2,4-Dichlorophenyl)-5-(4-hydroxyphenyl)-4-methyl-1H-pyrazole-3-carboxylic acid (4,4-difluorocyclohexyl)-amide

To a suspension of 5-(4-benzyloxyphenyl)-1-(2,4-dichlorophenyl)-4-methyl-1H-pyrazole-3-carboxylic acid (4,4-difluorocyclohexyl)amide (1.16 g, 2.03 mmol) in ethanol (20 ml) was added palladium on carbon (100 mg) and the reaction mixture hydrogenated with aid of a balloon for 5 hrs. Filtration, concentration and purification by chromatography (silica gel, heptane:EtOAc 50:50-EtOAc) afforded 0.84 g (88%) of the title compound as a pale yellow solid.

### Step G 5-[4-(tert-Butyldimethylsilanyloxy)-phenyl]-1-(2,4-dichlorophenyl)-4-methyl-1H-pyrazole-3-carboxylic acid (4,4-difluorocyclohexyl)amide

To a solution of 1-(2,4-dichlorophenyl)-5-(4-hydroxyphenyl)-4-methyl-1H-pyrazole-3-carboxylic acid (4,4-difluorocyclohexyl)amide (0.84 g, 1.79 mmol) in DMF (20 ml) was added imidazole (0.24 g, 3.60 mmol) followed by t-butylchlorodimethylsilane (0.54 g, 3.60 mmol). The reaction mixture was stirred at room temperature overnight, diluted with water and extracted with ether (×2). The combined organic extracts were washed with water, dried (Na₂SO₄), filtered and concentrated. Purification by flash chromatography (heptane:EtOAc gradient) afforded 0.85 g (80%) of the title compound as a colorless solid.

### Step H 4-Bromomethyl-5-[4-(tert-Butyldimethylsilanyloxy)-phenyl]-1-(2,4-dichlorophenyl)-1H-pyrazole-3-carboxylic acid (4,4-difluorocyclohexyl)amide

To a solution of 5-[4-(tert-butyldimethylsilanyloxy)phenyl]-1-(2,4-dichlorophenyl)-4-methyl-1H-pyrazole-3-carboxylic acid (4,4-difluorocyclohexyl)amide (0.81 g, 1.36 mmol) in 1,2-dichloroethane (20 ml) was added N-bromosuccinimide (0.0.36 g, 2.04 mmol) and a catalytic amount of AIBN. The reaction mixture was heated at reflux for 2 hrs, cooled and subjected to purification by chromatography (heptane:EtOAc gradient) affording 0.60 g (66%) of the title compound.

### Step I 5-[4-(tert-Butyldimethylsilanyloxy)phenyl]-1-(2,4-dichlorophenyl)-4-hydroxymethyl-1H-pyrazole-3-carboxylic acid (4,4-difluorocyclohexyl)amide

To a solution of 4-bromomethyl-5-[4-(tert-butyldimethylsilanyloxy)phenyl]-1-(2,4-dichloro-phenyl)-1H-pyrazole-3-carboxylic acid (4,4-difluorocyclohexyl)amide (0.52 g, 0.77 mmol) in acetone:water (10:10 ml) was added silver nitrate (0.46 g, 2.70 mmol) and the reaction mixture stirred at 60° C. overnight, cooled to rt, filtered and after addition of water extracted with DCM (×2). The combined organic extract was dried (Na₂SO₄), filtered and concentrated to afford 0.46 g (98%) of the title compound as a colorless solid.

### Step J 1-(2,4-Dichlorophenyl)-4-hydroxymethyl-5-(4-hydroxyphenyl)-1H-pyrazole-3-carboxylic acid (4,4-difluorocyclohexyl)amide

To a solution of 5-[4-(tert-butyldimethylsilanyloxy)phenyl]-1-(2,4-dichlorophenyl)-4-hydroxymethyl-1H-pyrazole-3-carboxylic acid (4,4-difluorocyclohexyl)amide (0.46 g, 0.75 mmol) in acetonitrile (40 ml) at 0° C. was added 48% HF (aq) (5.5 ml). The reaction mixture was allowed to reach room temperature and stirred overnight before being poured into ice-water, extracted with EtOAc (×2). The combined organic extracts were washed with water, dried (Na₂SO₄), filtered and concentrated.

### Step K 3,3,3-Trifluoro-propane-1-sulfonic acid 4-[2-(2,4-dichlorophenyl)-5-(4,4-difluoro-cyclohexylcarbamoyl)-4-hydroxymethyl-2H-pyrazol-3-yl]phenyl ester

To a magnetically stirred solution of 1-(2,4-dichlorophenyl)-4-hydroxymethyl-5-(4-hydroxy-phenyl)-1H-pyrazole-3-carboxylic acid (4,4-difluorocyclohexyl)amide (0.72 g, 1.45 mmol) in dry dichloromethane (35 ml) was added triethylamine (0.43 ml, 3.07 mmol) and the reaction mixture cooled to 0° C. 3,3,3-Trifluoro-1-propanesulfonyl chloride (317 mg, 1.62 mmol) was added and the reaction mixture stirred for 1 hour at 0° C., poured into water and extracted with DCM (×2). The combined organic extracts were dried (Na₂SO₄), filtered and concentrated. Flash chromatography (heptane:EtOAc 70:30-50:50) afforded 260 mg (27%) of the title compound as a colorless solid.

¹H NMR (CDCl₃): [symbol] 7.59-6.95 (8H, m), 5.15 (1H, m), 4.66 (2H, m), 4.15 (1H, m), 3.70-3.40 (2H, m), 3.00-2.70 (2H, m), 2.35-1.60 (8H, m). HPLC: 94.4%. MS (M+Na): 678

### Example 5

### 3,3,3-Trifluoropropane-1-sulfonic acid 4-[2-(2,4-dichlorophenyl)-4-hydroxymethyl-5-(piperidin-1-ylcarbamoyl)-2H-pyrazol-3-yl]phenyl ester

### Step A 1-(2,4-Dichloro-phenyl)-5-(4-hydroxyphenyl)-4-methyl-1H-pyrazole-3-carboxylic acid

To a solution of 1-(2,4-dichloro-phenyl)-5-(4-methoxyphenyl)-4-methyl-1H-pyrazole-3-carboxylic acid, Ex. 3, Step C (3.34 g, 8.85 mmol) in acetic acid was added 48% HBr (aq) (8.5 ml) dropwise and the reaction mixture refluxed overnight. After cooling to room temperature the reaction mixture was poured onto ice-water and extracted with EtOAc (×3). The combined organic extracts were washed with water, NaHCO₃ (aq) and brine. Drying (Na₂SO₄), filtration and concentration left 3.00 g (93%) of the title compound as a colorless solid.

### Step B 1-(2,4-Dichlorophenyl)-5-(4-hydroxyphenyl)-4-methyl-1H-pyrazole-3-carboxylic acid methyl ester

A solution of 1-(2,4-dichlorophenyl)-5-(4-hydroxyphenyl)-4-methyl-1H-pyrazole-3-carboxylic acid (0.36 g, 1.00 mmol) in 1.25 M HCl in methanol (10 ml) was refluxed for 1.5 hours. After cooling to room temperature, water was added and the product extracted with EtOAc (×3). The combined organic extracts were washed with water, dried (Na₂SO₄), filtered and concentrated. Flash chromatography (heptane:EtOAc 70:30-50:50) afforded 0.32 g (85%) of the title compound as a colorless solid.

### Step C 5-[4-(tert-Butyldimethyl-silanyloxy)phenyl]-1-(2,4-dichloro-phenyl)-4-methyl-1H-pyrazole-3-carboxylic acid methyl ester

To a solution of 1-(2,4-dichlorophenyl)-5-(4-hydroxyphenyl)-4-methyl-1H-pyrazole-3-carboxylic acid methyl ester (2.69 g, 7.13 mmol) in DMF (50 ml) was added imidazole (0.98 g, 14.3 mmol) followed by t-butyldichlorodimethylsilane (2.15 g, 14.3 mmol). The reaction mixture was stirred at room temperature overnight, diluted with water and extracted with ether (×2). The combined organic extracts were washed with water, dried (Na₂SO₄), filtered and concentrated. Purification by flash chromatography (heptane:EtOAc gradient) afforded 2.50 g (71%) of the title compound as a colorless solid.

### Step D 4-Bromomethyl-5-[4-(tert-butyldimethylsilanyloxy)-phenyl]-1-(2,4-dichlorophenyl)-1H-pyrazole-3-carboxylic acid methyl ester

To a solution of 5-[4-(tert-butyl-dimethylsilanyloxy)-phenyl]-1-(2,4-dichlorophenyl)-4-methyl-1H-pyrazole-3-carboxylic acid methyl ester (2.50 g, 5.08 mmol) in 1,2-dichloroethane (80 ml) was added N-bromosuccinimide (0.99 g, 5.60 mmol) and a catalytic amount of AIBN. The reaction mixture was heated at reflux for 2 hrs, cooled and subjected to purification by chromatography (heptane:EtOAc gradient) affording 2.60 g (89%) of the title compound.

### Step E 1-(2,4-Dichloro-phenyl)-4-hydroxymethyl-5-(4-hydroxy-phenyl)-1H-pyrazole-3-carboxylic acid methyl ester

To a suspension of 4-bromomethyl-5-[4-(tert-butyldimethyl-ilanyloxy)phenyl]-1-(2,4-dichloro-phenyl)-1H-pyrazole-3-carboxylic acid methyl ester (0.57 g, 1.00 mmol) in acetone:water (10:10 ml) was added silver nitrate (0.60 g, 3.52 mmol) and the reaction mixture stirred at 60° C. overnight, cooled to rt, filtered and after addition of water extracted with DCM (×2). The combined organic extracts were dried (Na₂SO₄), filtered and concentrated to afford 0.37 g (95%) of the title compound as a colorless solid.

### Step F 1-(2,4-Dichlorophenyl)-4-hydroxymethyl-5-(4-hydroxyphenyl)-1H-pyrazole-3-carboxylic piperidin-1-yl-amide

To a suspension of AlCl₃ (1.59 g, 11.9 mmol) in 1,2-dichloroethane (20 ml) at 0° C. was added 1-aminopiperidine (2.38 g, 23.8 mmol). The reaction mixture allowed to reach room temperature and a solution of 1-(2,4-dichlorophenyl)-4-hydroxymethyl-5-(4-hydroxyphenyl)-1H-pyrazole-3-carboxylic acid methyl ester (1.17 g, 2.98 mmol) in 1,2-dichloroethane (20 ml) added. The reaction mixture was heated 1 hour at 60° C., cooled to room temperature, poured into ice-water, and extracted with DCM (×3). The combined organic extracts were washed with water, dried (Na₂SO₄), filtered and concentrated. Flash chromatography (EtOAc) produced 1.00 g (73%) of the title compound as a pale yellow solid.

### Step G 3,3,3-Trifluoropropane-1-sulfonic acid 4-[2-(2,4-dichlorophenyl)-4-hydroxymethyl-5-(piperidin-1-ylcarbamoyl)-2H-pyrazol-3-yl]phenyl ester

To a magnetically stirred solution of 1-(2,4-dichlorophenyl)-4-hydroxymethyl-5-(4-hydroxy-phenyl)-1H-pyrazole-3-carboxylic piperidin-1-yl-amide (720 mg, 1.56 mmol) in dry dichloromethane (14 ml) was added triethylamine (0.49 ml, 3.50 mmol) and the reaction mixture cooled to 0° C. 3,3,3-Trifluoro-1-propanesulfonyl chloride (350 mg, 1.78 mmol) was added and the reaction mixture stirred for 2 hours at 0° C., poured into water and extracted with DCM (×2). The combined organic extracts were dried (Na₂SO₄), filtered and concentrated. Flash chromatography (heptane:EtOAc) afforded 380 mg (39%) of the title compound as a colorless solid.

¹H NMR (CDCl₃): [symbol] 7.49-7.22 (8H, m), 4.66 (2H, s), 3.57-3.30 (6H, m), 2.90-2.76 (2H, m), 2.01-1.96 (4H, m), 1.33-1.26 (2H, m). HPLC: 93.5%. MS (M+Na): 643

### Example 6

### Propane-1-sulfonic acid 4-[2-(2,4-dichlorophenyl)-5-(4,4-difluoro-cyclohexylcarbamoyl)-4-hydroxymethyl-2H-pyrazol-3-yl]phenyl ester

To a magnetically stirred solution of 1-(2,4-dichlorophenyl)-4-hydroxymethyl-5-(4-hydroxy-phenyl)-1H-pyrazole-3-carboxylic acid (4,4-difluorocyclohexyl)-amide, from Ex. 4, Step J (240 mg, 0.48 mmol) in dry dichloromethane (10 ml) was added triethylamine (0.14 ml, 1.00 mmol) and the reaction mixture cooled to 0° C. 1-Propanesulfonyl chloride (54 μl, 0.48 mmol) was added and the reaction mixture stirred for 1 hour at 0° C., poured into water and extracted with DCM (×2). The combined organic extracts were dried (Na₂SO₄), filtered and concentrated. Flash chromatography afforded 200 mg (69%) of the title compound as a colorless solid.

¹H NMR (CDCl₃): δ7.50-7.02 (8H, m), 4.66 (2H, s), 4.15 (1H, m), 3.60 (1H, broad s), 3.26 (2H, m), 2.20-1.70 (10H, m), 1.16 (3H, t). HPLC. 96.2%. MS (M+Na): 624.

### Example 7

### 3,3,3-Trifluoropropane-1-sulfonic acid 4-[2-(2,4-dichlorophenyl)-4-hydroxymethyl-5-(5-trifluoromethylpyridin-2-ylcarbamoyl)-2H-pyrazol-3-yl]phenyl ester

### Step A 1-(2,4-Dichlorophenyl)-5-(4-methoxyphenyl)-4-methyl-1H-pyrazole-3-carboxylic acid (5-trifluoromethylpyridin-2-yl)-amide

To a solution of 1-(2,4-dichlorophenyl)-5-(4-methoxyphenyl)-4-methyl-1H-pyrazole-3-carboxylic acid (1.00 g, 2.65 mmol) in DCM (20 ml) was added 5 drops of DMF followed by 5 ml oxalyl chloride. The reaction mixture was stirred for one hour at room temperature, concentrated and the crude acid chloride redissolved in DCM (20 ml). DMAP (0.98 g, 8.03 mmol) was added followed by 5-(trifluoromethyl)pyridine-2-amine (0.49 g, 3 mmol). The reaction mixture was stirred at room temperature overnight then water was added and the product extracted with DCM (×2). The combined organic extracts were dried (Na₂SO₄), filtered and concentrated. Flash chromatography (heptane-heptane:EtOAc 90:10) gave 0.67 g (49%) of the title compound as a colorless solid.

### Step B 1-(2,4-Dichlorophenyl)-5-(4-hydroxyphenyl)-4-methyl-1H-pyrazole-3-carboxylic acid (5-trifluoromethyl-pyridin-2-yl)-amide

To a solution of 1-(2,4-dichlorophenyl)-5-(4-methoxyphenyl)-4-methyl-1H-pyrazole-3-carboxylic acid (5-trifluoromethylpyridin-2-yl)amide (0.59 g, 1.13 mmol) in dichloromethane (20 ml) was added boron tribromide (0.54 ml, 5.66 mmol) at 0° C. The reaction mixture was stirred at this temperature for one hour, poured onto ice-water and extracted twice with DCM. Drying (Na₂SO₄), filtration and concentration of the combined extracts followed by flash chromatography (heptane:EtOAc gradient) afforded 0.55 g (96%) of the title compound as a colorless solid.

### Step C 5-[4-(tert-Butyldimethylsilanyloxy)phenyl]-1-(2,4-dichlorophenyl)-4-methyl-1H-pyrazole-3-carboxylic acid (5-trifluoromethylpyridin-2-yl)amide

To a solution of 1-(2,4-dichlorophenyl)-5-(4-hydroxyphenyl)-4-methyl-1H-pyrazole-3-carboxylic acid (5-trifluoromethylpyridin-2-yl)-amide (0.55 g, 1.08 mmol) in DCM (30 ml) was added imidazole (0.30 g, 4.33 mmol) followed by t-butyldichlorodimethylsilane (0.65 g, 4.33 mmol). The reaction mixture was stirred at room temperature overnight, diluted with water and extracted with DCM (×2). The combined organic extracts were washed with water, dried (Na₂SO₄), filtered and concentrated. Purification by flash chromatography (heptane:EtOAc gradient) afforded 0.62 g (92%) of the title compound as a colorless solid.

### Step D 4-Bromomethyl-5-[4-(tert-butyldimethylsilanyloxy)phenyl]-1-(2,4-dichlorophenyl)-1H-pyrazole-3-carboxylic acid (5-trifluoromethylpyridin-2-yl)amide

To a solution of 5-[4-(tert-butyldimethylsilanyloxy)phenyl]-1-(2,4-dichlorophenyl)-4-methyl-1H-pyrazole-3-carboxylic acid (5-trifluoromethylpyridin-2-yl)amide (0.62 g, 0.997 mmol) in 1,2-dichloroethane (20 ml) was added N-bromosuccinimide (0.21 g, 1.20 mmol) and a catalytic amount of AIBN. The reaction mixture was heated at reflux for 1 hr, cooled and concentrated to dryness. Used directly in the subsequent step.

### Step E 1-(2,4-Dichlorophenyl)-4-hydroxymethyl-5-(4-hydroxyphenyl)-1H-pyrazole-3-carboxylic acid (5-trifluoromethylpyridin-2-yl)amide

To a suspension of 4-bromomethyl-5-[4-(tert-butyldimethylsilanyloxy)phenyl]-1-(2,4-dichloro-phenyl)-1H-pyrazole-3-carboxylic acid (5-trifluoromethylpyridin-2-yl)amide (0.69 g, 1.00 mmol) in acetone:water (15:15 ml) was added silver nitrate (0.60 g, 3.50 mmol) and the reaction mixture stirred at 60° C. overnight, cooled to rt, filtered and after addition of water extracted with DCM (×2). The combined organic extracts were dried (Na₂SO₄), filtered and concentrated to afford 0.44 g (85%, two steps) of the title compound as a colorless solid after flash chromatography (heptane:EtOAc gradient).

### Step F 3,3,3-Trifluoropropane-1-sulfonic acid 4-[2-(2,4-dichlorophenyl)-4-hydroxymethyl-5-(5-trifluoromethylpyridin-2-ylcarbamoyl)-2H-pyrazol-3-yl]phenyl ester

To a magnetically stirred solution of 1-(2,4-dichlorophenyl)-4-hydroxymethyl-5-(4-hydroxy-phenyl)-1H-pyrazole-3-carboxylic acid (5-trifluoromethylpyridin-2-yl)-amide (0.44 g, 0.84 mmol) in dry dichloromethane (20 ml) was added triethylamine (0.13 ml, 0.92 mmol) and the reaction mixture cooled to 0° C. 3,3,3-Trifluoro-1-propane sulfonyl chloride (181 mg, 0.92 mmol) was added and the reaction mixture stirred for 30 min. at 0° C., poured into water and extracted with DCM (×2). The combined organic extracts were dried (Na₂SO₄), filtered and concentrated. Flash chromatography (heptane:EtOAc 90:10-80:20) afforded 350 mg (61%) of the title compound as a colorless solid.

¹H NMR (CDCl₃): [symbol] 9.68 (1H, s), 8.63 (1H, s), 8.57-8.50 (1H, m), 8.05-7.98 (1H, m), 7.50-7.20 (7H, m), 4.78-4.68 (2H, m), 4.44 (1H, t), 3.60-3.47 (2H, m), 2.92-2.70 (2H, m).

HPLC: 98.4%. MS (M+H): 683

### Example 8

### 3,3,3-Trifluoropropane-1-sulfonic acid 4-[2-(2,4-dichlorophenyl)-5-(2-hydroxycyclohexylcarbamoyl)-4-hydroxymethyl-2H-pyrazol-3-yl]phenyl ester

### Step A: 4-Bromomethyl-5-[4-(tert-butyl-dimethyl-silanyloxy)-phenyl]-1-(2,4-dichloro-phenyl)-1H-pyrazole-3-carboxylic acid methyl ester

To a solution of 5-[4-(tert-butyl-dimethylsilanyloxy)-phenyl]-1-(2,4-Dichloro-phenyl)-4-methyl-1H-pyrazole-3-carboxylic acid methyl ester, Ex. 5, Step C (3.70 g, 7.53 mmol) in 1,2-dichloroethane (120 ml) was added N-bromosuccinimide (1.51 g, 8.48 mmol) and a catalytic amount of AIBN. The reaction mixture was heated at reflux for one hour, cooled to room temperature, water was added and the product extracted with DCM (×2). The combined organic extracts were dried Na₂SO₄), filtered and concentrated.

Chromatography (heptane:EtOAc gradient) affording 4.15 g (96%) of the title compound as a colorless solid.

### Step B 1-(2,4-Dichlorophenyl)-4-hydroxymethyl-5-(4-hydroxyphenyl)-1H-pyrazole-3-carboxylic acid methyl ester

To a suspension of 4-bromomethyl-5-[4-(tert-butyldimethylsilanyloxy)phenyl]-1-(2,4-dichlorophenyl)-1H-pyrazole-3-carboxylic acid methyl ester (4.15 g, 7.27 mmol) in acetone:water (40:40 ml) was added silver nitrate (4.32 g, 25.4 mmol) and the reaction mixture stirred at 60° C. overnight, cooled to rt, filtered and after addition of water extracted with DCM (×2). The combined organic extracts were washed with brine, dried (Na₂SO₄), filtered and concentrated to afford 2.47 g (87%) of the title compound as a colorless solid after flash chromatography (heptane:EtOAc 50:50).

### Step C 1-(2,4-Dichlorophenyl)-4-hydroxymethyl-5-[4-(3,3,3-trifluoropropane-1-sulfonyloxy)phenyl]-1H-pyrazole-3-carboxylic acid methyl ester

To a solution of 1-(2,4-dichlorophenyl)-4-hydroxymethyl-5-(4-hydroxyphenyl)-1H-pyrazole-3-carboxylic acid methyl ester (980 mg, 2.50 mmol) in dichloromethane (10 ml) was added triethylamine (0.42 ml, 3.00 mmol) at 0° C. followed by 3,3,3-trifluoropropanesulfonyl chloride (589 mg, 3.00 mmol) in dichloromethane (10 ml). The reaction mixture was stirred at 0° C. for 2 hours, poured into water, extracted with dichloromethane (×3), the combined organic extracts dried (Na₂SO₄), filtered and concentrated. Flash chromatography (heptane:EtOAc 70:30-50:50) afforded 0.77 g (56%) of the product as a colorless solid.

### Step D 1-(2,4-Dichlorophenyl)-4-hydroxymethyl-5-[4-(3,3,3-trifluoro-propane-1-sulfonyloxy)phenyl]-1H-pyrazole-3-carboxylic acid

To a solution of 1-(2,4-dichlorophenyl)-4-hydroxymethyl-5-[4-(3,3,3-trifluoropropane-1-sulfonyloxy)phenyl]-1H-pyrazole-3-carboxylic acid methyl ester (0.77 g, 1.39 mmol) in MeOH:THF (10 ml: 10 ml) at 0° C. was added a solution of lithium hydroxide (0.23 g, 5.60 mmol) in water (10 ml). The reaction mixture was stirred at this temperature for 1.5 hrs, acidified with 1M HCl to pH 3 and the product extracted with EtOAc (×3). The combined organic extracts were washed with water, dried (Na₂SO₄), filtered and concentrated. Flash chromatography (heptane:EtOAc 50:50) afforded 0.70 g (93%) of the title compound as a colorless solid.

### Step E 3,3,3-Trifluoropropane-1-sulfonic acid 4-[2-(2,4-dichlorophenyl)-5-(2-hydroxy-cyclohexylcarbamoyl)-4-hydroxymethyl-2H-pyrazol-3-yl]phenyl ester

To a suspension of 1-(2,4-dichlorophenyl)-4-hydroxymethyl-5-[4-(3,3,3-trifluoropropane-1-sulfonyloxy)phenyl]-1H-pyrazole-3-carboxylic acid (0.70 g, 1.30 mmol) in dichloromethane (90 ml) was added cis-2-aminocyclohexanol hydrochloride (200 mg, 1.32 mmol) followed by triethylamine (0.37 ml, 2.63 mmol) and BOP (708 mg, 1.60 mmol). The reaction mixture was stirred at room temperature overnight, poured into ice-water and extracted with EtOAc (×3). The combined organic extracts were dried (Na₂SO₄), filtered and concentrated. Flash chromatography (heptane:EtOAc 50:50-EtOAc) and CH₂Cl₂:

MeOH 99: 1-95:5) afforded 200 mg (24%) of the title compound as a colorless solid.

HPLC: 89%. MS: 658 (M+Na).

### Example 9

### 3,33-Trifluoropropane-1-sulfonic acid 4-[5-cyclohexylcarbamoyl-2-(2,4-dichloro-phenyl-4-hydroxymethyl-2H-pyrazol-3-yl]phenyl ester

### Step A 1-(2,4-Dichlorophenyl-4-hydroxymethyl-5-(4-hydroxyphenyl)-1H-pyrazole-3-carboxylic acid cyclohexylamide

To a suspension of AlCl₃ (0.53 g, 4.00 mmol) in 1,2-dichloroethane (10 ml) at 0° C. was added cyclohexylamine (0.92 ml, 8.00 mmol). The reaction mixture allowed to reach room temperature and a solution of 1-(2,4-dichloro-phenyl)-4-hydroxymethyl-5-(4-hydroxyphenyl)-1H-pyrazole-3-carboxylic acid methyl ester, Ex. 8, Step B (0.39 g, 1.00 mmol) in 1,2-dichloroethane (10 ml) added. The reaction mixture was heated 1 hour at 60° C., cooled to room temperature, poured into ice-water, and extracted with EtOAc (×2). The combined organic extracts were washed with brine, dried (Na₂SO₄), filtered and concentrated to give 450 mg (97%) of the product as a colorless solid used directly in the next step

### Step B 3,3,3-Trifluoropropane-1-sulfonic acid 4-[5-cyclohexylcarbamoyl-2-(2,4-dichloro-phenyl)-4-hydroxymethyl-2H-pyrazol-3-yl]-phenyl ester

To a magnetically stirred solution of 1-(2,4-dichlorophenyl)-4-hydroxymethyl-5-(4-hydroxyphenyl)-1H-pyrazole-3-carboxylic acid cyclohexylamide (450 mg, 0.98 mmol) in dry dichloromethane (20 ml) at 0° C. was added triethylamine (0.15 ml, 1.07 mmol) followed by 3,3,3-trifluoro-1-propane sulfonyl chloride (210 mg, 1.07 mmol). The cooling bath was removed and the reaction mixture stirred for 2 hours at room temperature. The reaction mixture was concentrated and purified by flash chromatography (heptane:EtOAc 80:20-70: 30-50:50) to afford 380 mg (62%) of the title compound as a colorless solid.

¹H NMR (CDCl₃): [symbol] 7.497.22 (7H, m), 7.02 (1H, m), 4.65 (2H, s), 4.03-3.99 (1H, broad s), 3.99-3.49 (2H, m), 2.89-2.76 (2H, m), 2.08-2.04 (2H, m), 1.84-1.25 (9H, m). HPLC: 97.5%. MS (M+Na): 643

### Example 10

### N-cyclohexyl-1-(2,4-dichlorophenyl)-4-hydroxymethyl-5-[4-(3,3,3-trifluoropropoxy)phenyl]-1H-pyrazole-3-carboxamide

### Step A Lithium 1-(4-Benzyloxyphenyl)-3-ethoxycarbonyl-2-methyl-3-oxopropen-1-ol

To a solution of 4-benzyloxypropiophenone, Ex. 4, Step A (50 g, 0.2083 mol) in dry THF (500 ml) at 0° C. was added LiHMDS (1M solution in THF, 208.3 ml) dropwise over a period of 1 h under N₂ atm. The reaction mixture was stirred at 0° C. for 1 hr. Diethyl oxalate (33.49 g, 0.2296 mol) was added dropwise. The reaction mixture was allowed to warm to RT and stirred at RT for 16 hrs under N₂ atm. The reaction mixture was concentrated in a rotary evaporator at RT. Dry diethyl ether (1 L) was added to the residue and the solid was collected by filtration, washed with dry ether, and dried under vacuum to yield lithium salt of the diketoester (50 g) as yellow solid.

### Step B 4-(4-Benzyloxyphenyl)-4-[(2,4-dichlorophenyl)hydrazono]-3-methyl-2-oxo-butyric acid ethyl ester

A mixture of lithium salt from step 2 (50 g, 0.1461 mol) and 2,4-dichlorophenyl hydrazine hydrochloride (34.33 g, 0.1608 mol) in ethanol (500 ml) was stirred at RT under N₂ atm for 18 hrs. The precipitate was filtered, washed with dry ether and dried under vacuum to yield hydrazone intermediate (35 g).

### Step C Ethyl 5-[4-(benzyloxy)phenyl]-1-(2,4-dichlorophenyl)-4-methyl-1H-pyrazole-3-carboxylate

Hydrazone intermediate (35 g) was dissolved in acetic acid (250 ml) and heated under reflux for 18 hrs. Reaction mixture was poured into cold water (2 L) and extracted with ethyl acetate (2×500 ml). Combined organic layer was washed with water, sat. NaHCO₃ and brine, dried over Na₂SO₄, concentrated and purified by column chromatography over silica gel using 20% ethyl acetate in petroleum ether as eluent to yield the title compound (22 g) as yellow solid.

### Step D Ethyl 1-(2,4-dichlorophenyl)-5-(4-hydroxyphenyl)-4-methyl-1H-pyrazole-3-carboxylate

Ethyl 5-[4-(benzyloxy)phenyl]-1-(2,4-dichlorophenyl)-4-methyl-1H-pyrazole-3-carboxylate (4.82 g, 10 mmol) was dissolved in 80 ml HBr (33% in acetic acid) and stirred overnight at room temperature with exclusion of light. The solvents were evaporated and the residue coevaporated twice with ethanol. The residue was dissolved in EtOAc and washed with water basified with triethylamine and then brine. The organic layer was dried over Na₂SO₄ and evaporated to give ethyl 1-(2,4-dichlorophenyl)-5-(4-hydroxyphenyl)-4-methyl-1H-pyrazole-3-carboxylate (4.54 g) as a brown, viscous oil of sufficient purity for the next step.

¹H NMR (500 MHz, CDCl₃) [symbol] 7.45-7.23 (m, 3H), 6.98 (d, J=8.7 Hz, 2H), 6.80 (d, J=8.7 Hz, 2H), 4.43 (q, J=7.1 Hz, 2H), 2.33 (s, 3H), 1.40 (t, J=7.1 Hz, 3H).

### Step E Ethyl 1-(2,4-dichlorophenyl)-4-methyl-5-[4-(3,3,3-trifluoropropoxy)phenyl]-1H-pyrazole-3-carboxylate

Ethyl 1-(2,4-dichlorophenyl)-5-(4-hydroxyphenyl)-4-methyl-1H-pyrazole-3-carboxylate (1.51 g, 3.87 mmol), 3,3,3-trifluoro-1-propanol (2.21 g, 19.4 mmol) and triphenylphosphine (5.08 g, 19.4 mmol) were dissolved in anhydrous THF (20 ml). Then DEAD (3.2 ml of a ca. 40% solution in toluene, d=0.95, 7.76 mmol) was added. The resulting mixture was stirred at room temperature for 20 h, then an additional portion of DEAD (3.2 ml of a ca. 40% solution in toluene, d=0.95, 7.76 mmol) was added and stirring continued for 7 h, then again DEAD (1.6 ml of a ca. 40% solution in toluene, d=0.95, 3.88 mmol) was added and stirring continued for 16 h. The solvents were evaporated, the residue dissolved in 20 ml EtOAc and 80 ml of hexanes were added. Precipitation occurs. The resulting mixture was sonicated for ca. 5 min, the solid was filtered off and washed with hexanes/EtOAc 4:1. The combined filtrates were evaporated and the residue purified by column chromatography (silica gel, hexanes/EtOAc, 10-20%) to yield ethyl 1-(2,4-dichlorophenyl)-4-methyl-5-[4-(3,3,3-trifluoropropoxy)phenyl]-1H-pyrazole-3-carboxylate (1.81 g, 3.34 mmol, 86%) as a yellowish foam which contains ca. 10% of diethyl hydrazine-1,2-dicarboxylate, which does not interfere with the next transformation.

¹H NMR (400 MHz, CDCl₃) [symbol] 7.35-7.22 (m, 3H), 7.00 (d, J=8.7 Hz, 2H), 6.81 (d, J=8.7 Hz, 2H), 4.43 (q, J=7.1 Hz, 2H), 4.18-4.13 (m, 2H), 2.65-2.55 (m, 2H), 2.30 (s, 3H), 1.40 (t, J=7.1 Hz, 3H)

### Step F Ethyl 1-(2,4-dichlorophenyl)-4-hydroxymethyl-5-[4-(3,3,3-trifluoropropoxy)phenyl]-1H-pyrazole-3-carboxylate

To a solution of ethyl 1-(2,4-dichlorophenyl)-5-[4-(3-fluoropropoxy)phenyl]-4-methyl-1H-pyrazole-3-carboxylate (260 mg, 0.48 mmol) in CCl₄ (10 ml) was added N-bromosuccinimide (94 mg, 0.53 mmol) and 2,2′-isobutyronitrile (15 mg, 0.091 mmol). The resulting mixture was heated under reflux for 2 h, then stirred at room temperature overnight. The formed precipitate was filtered and the filtrate evaporated. The residue was again dissolved in CCl₄ (10 ml) and N-bromosuccinimide (30 mg, 0.168 mmol) and 2,2′-azobisisobutyronitrile (10 mg, 0.060 mmol) were added. The reaction mixture was heated under reflux for 2 h, cooled to room temperature, filtered and the filtrate evaporated. The oily residue was dissolved in acetone (6 ml) and water (6 ml). Silver nitrate (337 mg, 1.98 mmol) was added and the resulting mixture warmed to 60 C for 2 h. The reaction mixture was cooled to room temperature, diluted with water and extracted with dichloromethane three times. The combined organic layers were dried over MgSO₄ and evaporated. The residue was purified by column chromatography (silica gel, hexanes/EtOAc, 20-40%) to yield ethyl 1-(2,4-dichlorophenyl)-4-hydroxymethyl-5-[4-(3,3,3-trifluoropropoxy)phenyl]-1H-pyrazole-3-carboxylate (139 mg, 0.276 mmol, 56%) as an oil.

¹H NMR (400 MHz, CDCl₃) [symbol] 7.35-7.22 (m, 3H), 7.07 (d, J=8.7 Hz, 2H), 6.79 (d, J=8.7 Hz, 2H), 4.60 (s, 2H), 4.45 (q, J=7.1 Hz, 2H), 4.14-4.10 (m, 2H), 3.84 (s, 1H), 2.62-2.51 (m, 2H), 1.40 (t, J=7.1 Hz, 3H)

### Step G N-cyclohexyl-1-(2,4-dichlorophenyl)-4-hydroxymethyl-5-[4-(3,3,3-trifluoropropoxy)phenyl]-1H-pyrazole-3-carboxamide

Cyclohexylamine (198 mg, 2.0 mmol) was dissolved in toluene (5 ml) and cooled in an ice-bath. Trimethylaluminum (1 ml, 2M in toluene, 2 mmol) was added. After completed addition the reaction mixture was warmed to room temperature and stirred for 1 h. This mixture was then added to ethyl 1-(2,4-dichlorophenyl)-4-hydroxymethyl-5-[4-(3,3,3-trifluoropropoxy)phenyl]-1H-pyrazole-3-carboxylate (129 mg, 0.256 mmol) and warmed to 50° C. for 1 h, then cooled in an ice-bath. 1N HCl was added to the reaction mixture. After gas evolution had ceased, EtOAc was added and the phases separated. The organic layer was washed with 1N HCl and brine, dried over MgSO₄ and evaporated. The residue was purified by column chromatography (silica gel, hexanes/EtOAc, 20-40%) to yield N-cyclohexyl 1-(2,4-dichlorophenyl)-4-hydroxymethyl-5-[4-(3,3,3-trifluoropropoxy)phenyl]-1H-pyrazole-3-carboxamide (122 mg, 0.219 mmol, 85%) as a colorless foam.

¹H NMR (400 MHz, CDCl₃) [symbol] 7.39-7.20 (m, 3H), 7.03-6.98 (m, 3H), 6.78 (d, J=8.7 Hz, 2H), 5.39 (s, 1H), 4.58-4.56 (m, 2H), 4.14-4.10 (m, 2H), 3.97-3.87 (m, 1H), 2.62-2.51 (m, 2H), 2.10-1.94 (m, 2H), 1.74-1.70 (m, 2H), 1.63-1.57 (m, 1H), 1.42-1.32 (m, 2H), 1.28-1.09 (m, 3H. HRMS Calcd for [C₂₆H₂₆Cl₂F₃N₃O₃+H]⁺: 556.1832. Found: 556.1351.

### Example 11

### 1-(2,4-Dichlorophenyl)-N-(4,4-difluorocyclohexyl-4-hydroxymethyl-5-[4-(3,3,3-trifluoropropoxy)phenyl]-1H-pyrazole-3-carboxamide

4,4-Difluorocyclohexylamine (370 mg, 2.73 mmol) was dissolved in toluene (5 ml) and cooled in an ice-bath. Trimethylaluminum (1.35 ml, 2M in toluene, 2.70 mmol) was added. After completed addition the reaction mixture was warmed to room temperature and stirred for 90 min. This mixture was then added to ethyl 1-(2,4-dichlorophenyl)-4-hydroxymethyl-5-[4-(3,3,3-trifluoropropoxy)phenyl]-1H-pyrazole-3-carboxylate (139 mg, 0.276 mmol) and warmed to 50° C. for 90 min, then cooled in an ice-bath. 1N HCl was added to the reaction mixture. After gas evolution had ceased, EtOAc was added and the phases separated. The organic layer was washed with 1N HCl and brine, dried over MgSO₄ and evaporated. The residue was purified by column chromatography (silica gel, hexanes/EtOAc, 20-40%) to yield 1-(2,4-dichlorophenyl)-N-(4,4-difluorocyclohexyl)-4-hydroxymethyl-5-[4-(3,3,3-trifluoropropoxy)phenyl]-1H-pyrazole-3-carboxamide (126 mg, 0.212 mmol, 77%) as a colorless foam.

¹H NMR (500 MHz, CDCl₃) [symbol] 7.41-7.20 (m, 3H), 7.04-7.00 (m, 3H), 6.80 (d, J=8.7 Hz, 2H), 5.18 (d, J=6.8 Hz, 1H), 4.58 (d, J=6.8 Hz, 2H), 4.16-4.05 (m, 3H), 2.64-2.52 (m, 2H), 2.16-2.02 (m, 4H), 1.96-1.79 (m, 2H), 1.70-1.61 (m, 2H). HRMS Calcd for [C₂₆H₂₄Cl₂F₅N₃O₃+H]⁺: 592.1193. Found: 592.1170.

### Example 12

### N-cyclohexyl 1-(2,4-dichlorophenyl-4-[(dimethylamino)methyl]-5-[4-(3-fluoropropoxy)phenyl]-1H-pyrazole-3-carboxamide

### Step A Ethyl 1-(2,4-dichlorophenyl)-5-[4-(3-fluoropropoxy)phenyl]-4-hydroxymethyl-1H-pyrazole-3-carboxylate

To a solution of ethyl 1-(2,4-dichlorophenyl)-5-[4-(3-fluoropropoxy)phenyl]-4-methyl-1H-pyrazole-3-carboxylate (2.09 g, 4.63 mmol) in CCl₄ (40 ml) was added N-bromosuccinimide (1.07 g, 6 mmol) and the resulting mixture was boiled under reflux, then 2,2′-azobisisobutyronitrile (228 mg, 1.39 mmol) was added. Boiling under reflux was continued for 45 min, then the mixture was cooled to room temperature, filtered and the filtrate evaporated. The oily residue was dissolved in acetone (80 ml) and warmed to 60° C.

A solution of silver nitrate (2.6 g, 15.3 mmol) in water (20 ml) was added and the resulting mixture warmed to 60° C. for 14 h. Brine was added and after 15 min the reaction mixture cooled to room temperature. The formed precipitate was filtered off and the filtrate reduced to the aqueous phase. The aqueous phase was diluted with water and extracted with dichloromethane three times. The combined organic layers were dried over Na₂SO₄ and evaporated. The residue was purified by column chromatography (silica gel, hexanes/EtOAc, 25-40%) to yield ethyl 1-(2,4-dichlorophenyl)-5-[4-(3-fluoropropoxy)phenyl]-4-hydroxymethyl-1H-pyrazole-3-carboxylate (1.55 g, 2.23 mmol, 72%) as a colorless foam.

¹H NMR (400 MHz, CDCl₃) [symbol] 7.38-7.22 (m, 3H), 7.02 (d, J=8.7 Hz, 2H), 6.81 (d, J=8.7 Hz, 2H), 4.62-4.53 (m, 4H), 4.48 (q, J=7.1 Hz, 2H), 4.07-4.05 (m, 2H), 3.83 (t, J=7.2 Hz, 1H), 2.18-2.08 (m, 2H), 1.45 (t, J=7.1 Hz, 3H)

### Step B N-cyclohexyl 1-(2,4-dichlorophenyl)-5-[4-(3-fluoropropoxy)phenyl]-4-hydroxymethyl-1H-pyrazole-3-carboxamide

Cyclohexylamine (520 mg, 5.24 mmol) was dissolved in toluene (5 ml) and cooled in an ice-bath. Triethylaluminium (2.5 ml, 2M in heptanes, 5 mmol) was added. After completed addition the reaction mixture was warmed to room temperature and stirred for 30 min. This mixture was then added to 1-(2,4-dichlorophenyl)-5-[4-(3-fluoropropoxy)phenyl]-4-hydroxymethyl-1H-pyrazole-3-carboxylate (300 mg, 0.64 mmol) and warmed to 50 C for 1 h, then cooled in an ice-bath. Water, then 1N HCl was added to the reaction mixture. After gas evolution had ceased, EtOAc was added and the phases separated. The organic layer was washed with brine, dried over Na₂SO₄ and evaporated. The residue was purified by column chromatography (silica gel, hexanes/EtOAc, 20-25%) to yield N-cyclohexyl 1-(2,4-dichlorophenyl)-5-[4-(3-fluoropropoxy)phenyl]-4-hydroxymethyl-1H-pyrazole-3-carboxamide (190 mg, 0.365 mmol, 57%) as a colorless solid.

¹H NMR (400 MHz, CDCl₃) [symbol] 7.40-7.21 (m, 3H), 7.03-6.96 (m, 3H), 6.80 (d, J=8.7 Hz, 2H), 5.38 (t, J=7.2 Hz, 1H), 4.67-4.54 (m, 4H), 4.07-4.03 (m, 2H), 3.93-3.96 (m, 1H), 2.17-2.10 (m, 2H), 2.09-2.02 (m, 2H), 1.77-1.73 (m, 2H), 1.65-1.61 (m, 1H), 1.47-1.37 (m, 2H), 1.34-1.16 (m, 3H). HRMS Calcd for [C₂₆H₂₈Cl₂FN₃O₃+H]⁺: 520.1570. Found: 520.1593.

### Example 13

### N-cyclohexyl 1-(2,4-dichlorophenyl)-4-[(dimethylamino)methyl]-5-[4-(3-fluoropropoxy)phenyl]-1H-pyrazole-3-carboxamide

### Step A Ethyl 1-(2,4-dichlorophenyl)-5-[4-(3-fluoropropoxy)phenyl]-4-formyl-1H-pyrazole-3-carboxylate

Ethyl 1-(2,4-dichlorophenyl)-5-[4-(3-fluoropropoxy)phenyl]-4-formyl-1H-pyrazole-3-carboxylate (203 mg, 0.436 mmol) was isolated as side product in the synthesis of ethyl 1-(2,4-dichlorophenyl)-5-[4-(3-fluoropropoxy)phenyl]-4-hydroxymethyl-1H-pyrazole-3-carboxylate.

¹H NMR (400 MHz, CDCl₃) [symbol] 10.50 (s, 1H), 7.40-7.23 (m, 3H), 7.17 (d, J=8.7 Hz, 2H), 6.80 (d, J=8.7 Hz, 2H), 4.68-4.52 (m, 4H), 4.48 (q, J=7.1 Hz, 2H), 4.07-4.03 (m, 2H), 2.20-2.06 (m, 2H), 1.43 (t, J=7.1 Hz, 3H).

### Step B Ethyl 1-(2,4-dichlorophenyl)-4-[(dimethylamino)methyl]-5-[4-(3-fluoropropoxy)phenyl]-1H-pyrazole-3-carboxylate

To a solution of ethyl 1-(2,4-dichlorophenyl)-5-[4-(3-fluoropropoxy)phenyl]-4-formyl-1H-pyrazole-3-carboxylate (203 mg, 0.436 mmol) in dimethylformamide (8 ml) was added dimethylamine hydrochloride (66 mg, 0.81 mmol) and the resulting mixture was stirred at room temperature for 10 min. Then polymer bound triacetoxyborohydride (MP-triacetoxyborohydride, Argonaut, 620 mg, loading=2.32 g/mmol, 1.44 mmol) was added. The reaction mixture was stirred at room temperature for 90 min, then more dimethylamine hydrochloride (96 mg, 1.18 mmol) was added and the reaction mixture stirred overnight at room temperature. The resin was filtered off and thoroughly washed with EtOAc and water. To the filtrate was added sat. NaHCO₃ and the phases were separated. The organic layer was washed with sat. NaHCO₃, dried over Na₂SO₄ and evaporated to yield ethyl 1-(2,4-dichlorophenyl)-4-[(dimethylamino)methyl]-5-[4-(3-fluoropropoxy)phenyl]-1H-pyrazole-3-carboxylate (219 mg, 0.44 mmol, quant.) as a yellow oil of sufficient purity for the next step.

¹H NMR (400 MHz, CDCl₃) [symbol] 7.33-7.19 (m, 3H), 7.16 (d, J=8.7 Hz, 2H), 6.76 (d, J=8.7 Hz, 2H), 4.65-4.49 (m, 2H), 4.39 (q, J=7.1 Hz, 2H), 4.03-4.00 (m, 2H), 3.54 (s, 2H), 2.14-2.04 (m, 8H), 1.36 (t, J=7.1 Hz, 3H).

### Step C N-cyclohexyl 1-(2,4-dichlorophenyl)-4-[(dimethylamino)methyl]-5-[4-(3-fluoropropoxy)phenyl]-1H-pyrazole-3-carboxamide

Cyclohexylamine (176 mg, 1.77 mmol) was dissolved in toluene (5 ml), then triethylaluminium (0.8 ml, 2M in heptanes, 1.6 mmol) was added. After completed addition the reaction mixture was stirred for 10 min. This mixture was then added to ethyl 1-(2,4-dichlorophenyl)-4-[(dimethylamino)methyl]-5-[4-(3-fluoropropoxy)phenyl]-1H-pyrazole-3-carboxylate (219 mg, 0.44 mmol, quant.) and warmed to 50° C. overnight. After cooling to room temperature water was added to the reaction mixture. After gas evolution had ceased, ether and water were added and the phases separated. The organic layer was washed with brine, dried over Na₂SO₄ and evaporated. The residue was purified by column chromatography (silica gel, dichloromethane/MeOH/triethylamine, 99:1:0.5 to 95:5:0.5) to yield N-cyclohexyl 1-(2,4-dichlorophenyl)-4-[(dimethylamino)methyl]-5-[4-(3-fluoropropoxy)phenyl]-1H-pyrazole-3-carboxamide (180 mg, 0.328 mmol, 74%) as a colorless solid.

¹H NMR (400 MHz, CDCl₃) [symbol] 9.71 (s, 1H), 7.33-7.19 (m, 3H), 7.03 (d, J=8.7 Hz, 2H), 6.80 (d, J=8.7 Hz, 2H), 4.68-4.53 (m, 2H), 4.06-4.02 (m, 2H), 4.01-3.92 (m, 1H), 3.42 (s, 2H), 2.20-2.00 (m, 10H), 1.73-1.60 (m, 3H), 1.46-1.35 (m, 2H), 1.22-1.11 (m, 3H). HRMS Calcd for [C₂₈H₃₃Cl₂FN₄O₂+H]⁺: 547.2043. Found: 547.2057.

### Example 14

### 3,3,3-Trifluoropropane-1-sulfonic acid 4-[5-cyclohexylcarbamoyl-2-(2,4-dichloro-phenyl)-4-dimethylaminomethyl-2H-pyrazol-3-yl]phenyl ester

### Step A

### 4-Bromomethyl-1-(2,4-dichlorophenyl)-5-(4-methoxyphenyl)-1H-pyrazole-3-carboxylic acid methyl ester

To a magnetically stirred solution of 1-(2,4-dichlorophenyl)-4-methyl-5-(4-methoxyphenyl)-1H-pyrazole-3-carboxylic acid methyl ester, prepared as described in Ex 1, Step A (7.18 g, 18.3 mmol) in 1,2-dichloroethane (400 ml) was added N-bromosuccinimide (3.60 g, 20.2 mmol) and 2,2′-azoisobutyronitrile (250 mg). The resulting mixture was heated at reflux for one hour, concentrated and purified by flash chromatography (heptane:EtOAc gradient) to give 8.29 g (96%) of the title compound as a colorless solid.

### Step B

### 1-(2,4-Dichloro-phenyl)-4-dimethylaminomethyl-5-(4-methoxy-phenyl)-1H-pyrazole-3-carboxylic acid methyl ester

To a solution of 4-bromomethyl-1-(2,4-dichlorophenyl)-5-(4-methoxyphenyl)-1H-pyrazole-3-carboxylic acid methyl ester (1.21 g, 2.57 mmol) in DMF (20 ml) was added dimethylamine (20 ml, 40% in water). The reaction was stirred at room temperature for 3 hours when TLC showed complete conversion of starting material. The reaction mixture was concentrated and subjected to flash chromatography (heptane:EtOAc gradient) to afford 1.11 g (100%) of the product as a colorless solid.

### Step C

### 1-(2,4-Dichloro-phenyl)-4-dimethylaminomethyl-5-(4-methoxy-phenyl)-1H-pyrazole-3-carboxylic acid cyclohexylamide

To a suspension of aluminum trichloride (1.36 g, 10.24 mmol) in 1,2-dichloroethane (25 ml) at 0° C. was added cyclohexylamine (2.4 ml, 20.5 mmol). The reaction mixture was allowed to reach room temperature, stirred for 10 min. and a solution of 1-(2,4-dichlorophenyl)-4-dimethylaminomethyl-5-(4-methoxy-phenyl)-1H-pyrazole-3-carboxylic acid methyl ester (1.11 g, 2.56 mmol) in 1,2-dichloroethane (25 ml) was added. The reaction mixture was heated at 60° C. for one hour, cooled to room temperature, poured into water and extracted with DCM (×3). The combined organic extracts were washed with brine, dried (Na₂SO₄), filtered and concentrated. Flash chromatography (Heptane:EtOAc gradient) afforded 1.20 g (93%) of the title compound as a colorless solid.

### Step D

### 1-(2,4-Dichlorophenyl)-4-dimethylaminomethyl-5-(4-hydroxyphenyl)-1H-pyrazole-3-carboxylic acid cyclohexylamide

To as solution of 1-(2,4-dichlorophenyl)-4-dimethylaminomethyl-5-(4-methoxy-phenyl)-1H-pyrazole-3-carboxylic acid cyclohexylamide (1.20 g, 2.40 mmol) in dichloromethane (20 ml) at was added BBr₃ (0.90 ml, 9.60 mmol) at 0° C. The reaction mixture was stirred for 2 hrs at room temperature, poured into water and the product extracted with DCM (×3). The organic extract was dried (Na₂SO₄), filtered, concentrated and purified by flash chromatography (DCM-DCM:MeOH 95:5-DCM:MeOH 90:10) afforded 1.00 g (86%) of the product as a colorless solid.

### Step E

3,3,3-Trifluoropropane-1-sulfonic acid 4-[5-cyclohexylcarbamoyl-2-(2,4-dichloro-phenyl)-4-dimethylaminomethyl-2H-pyrazol-3-yl]-phenyl ester

To a magnetically stirred solution of 1-(2,4-dichlorophenyl)-4-dimethylaminomethyl-5-(4-hydroxy-phenyl)-1H-pyrazole-3-carboxylic acid cyclohexylamide (1.00 g, 2.05 mmol) in dry dichloromethane (20 ml) at 0° C. was added triethylamine (0.33 ml, 2.40 mmol) followed by 3,3,3-trifluoro-1-propane sulfonyl chloride (471 mg, 2.40 mmol). The cooling bath was removed and the reaction mixture stirred for 1.5 hours at room temperature. The reaction mixture was concentrated and purified by flash chromatography (heptane:EtOAc gradient) to afford 250 mg (19%) of the title compound as a colorless solid. HPLC: 94% MS: 647 (M+H).

### Example 15

### 4-[1-(2-chlorophenyl)-3-[(cyclohexylamino)carbonyl]-4-(hydroxymethyl)-1H-pyrazol-5-yl]phenyl 3,3,3-trifluoropropane-1-sulfonate

### Step A: Ethyl 3-[4-(benzyloxy)phenyl]-2-methyl-3-oxopropanoate lithium salt

Para-benzyloxypropiophenone (3.84 g, 15.98 mmol) in dry THF (30 ml) was added to a solution of lithium bis(trimethylsilyl)amide (17.6 ml, 1M in hexanes) in diethyl ether (100 ml) at −78° C., under N₂ (g). The reaction was continued at −78° C., under N₂ (g) for 1 hour. Ethyl oxalate (2.40 ml, 17.74 mmol) was added. The reaction was continued at room temperature for 20 hours. The mixture was filtered and the filtrate washed with THF/diethyl ether 1:5 and diethyl ether and dried at reduced pressure (2.85 g crude).

### Step B: Ethyl 5-[4-(benzyloxy)phenyl]-1-(2-chlorophenyl-4-methyl-1H-pyrazole-3-carboxylate

Ethyl 3-[4-(benzyloxy)phenyl]-2-methyl-3-oxopropanoate lithium salt (2.84 g, crude) and (2-chlorophenyl)hydrazine hydrochloride (1.62 g, 9.02 mmol) were mixed in ethanol (50 ml) and reacted at room temperature for 19 hours. The solvent was evaporated, the mixture suspended in acetic acid (40 ml) and reacted at 100° C. for 7 hours. Water and DCM were added, the phases were separated and the organic phase evaporated. The product was purified further by flash chromatography (SiO₂, heptanes/ethyl acetate, product came at 30% ethyl acetate) (1.91 g, 52% for two steps).

¹H NMR (399.964 MHz) [symbol] 7.40-7-20 (m, 9H), 7.06 (d, 2H), 6.86 (d, 2H), 4.97 (s, 2H), 4.42 (q, 2H), 2.33 (s, 3H), 1.39 (t, 3H). MS m/z 447, 449 (M+H)⁺.

### Step C: Ethyl 1-(2-chlorophenyl)-5-(4-hydroxyphenyl)-4-methyl-1H-pyrazole-3-carboxylate

Ethyl 5-[4-(benzyloxy)phenyl]-1-(2-chlorophenyl)-4-methyl-1H-pyrazole-3-carboxylate (802 mg, 1.79 mmol) was dissolved in HBr in acetic acid (33%, 10 ml) and reacted at room temperature for 1 hour. The mixture was cooled to 0° C., ethanol was added and the mixture stirred for 1 hour. The solvent was evaporated. Methanol was added, the mixture neutralised with NaHCO₃ (5%, aq) and the solvent evaporated. Water and DCM were added. The phases separated and the organic phase evaporated (620 mg, crude).

¹H NMR (399.964 MHz) [symbol] 7.50-7.30 (br, 1H), 7.32-7.12 (m, 4H), 6.89 (d, 2H), 6.66 (d, 2H), 4.32 (q, 2H), 2.28 (s, 3H), 1.26 (t, 3H). MS m/z 357, 359 (M+H)⁺.

### Step D: Ethyl 5-(4-{[tert-butyl(dimethyl)silyl]oxy}phenyl)-1-(2-chlorophenyl-4-methyl-1H-pyrazole-3-carboxylate

Tert-butyl(chloro)dimethylsilane (1.03 g, 6.86 mmol) was added to a mixture of ethyl 1-(2-chlorophenyl)-5-(4-hydroxyphenyl)-4-methyl-1H-pyrazole-3-carboxylate (620 mg, crude) and TEA (1.21 ml) in DCM (20 ml). The reaction was continued at room temperature for 16 hours. Water and DCM were added. The phases were separated and the organic phase evaporated (901 mg, crude). MS m/z 471, 473 (M+H)⁺.

### Step E: Ethyl 4-(bromomethyl)-5-(4-{[tert-butyl(dimethyl)silyl]oxy}phenyl)-1-(2-chlorophenyl)-1H-pyrazole-3-carboxylate

N-Bromosuccinimide (527 mg, 2.96 mmol) and 2,2′-azobisisobutyronitrile (17.5 mg, 0.10 mmol) were added to a mixture of ethyl 5-(4-{[tert-butyl(dimethyl)silyl]oxy}phenyl)-1-(2-chlorophenyl)-4-methyl-1H-pyrazole-3-carboxylate (901 mg, crude) in 1,2-dichloroethane (20 ml). The mixture was refluxed for 2 hours. Water and DCM were added, the phases separated and the organic phase evaporated (1.18 g, crude). MS m/z 549, 551, 553 (M)⁺.

### Step F: Ethyl 1-(2-chlorophenyl)-4-(hydroxymethyl)-5-(4-hydroxyphenyl)-1H-pyrazole-3-carboxylate

Silver nitrate (1.48 g, 8.72 mmol) was added to a mixture of ethyl 4-(bromomethyl)-5-(4-{[tert-butyl(dimethyl)silyl]oxy}phenyl)-1-(2-chlorophenyl)-1H-pyrazole-3-carboxylate (1.18 g, crude) in water/acetone (1:1, 80 ml). The mixture was heated to 60° C. for 16 hours. The mixture was cooled to room temperature. DCM was added, the phases were separated and the organic phase evaporated (756 mg, crude). MS m/z 373, 375 (M+H)⁺.

### Step G: Ethyl 1-(2-chlorophenyl)-4-(hydroxymethyl)-5-(4-{[(3,3,3-trifluoropropyl)sulfonyl]oxy}phenyl)-1H-pyrazole-3-carboxylate

3,3,3-Trifluoropropane-1-sulfonyl chloride (499 mg, 2.54 mmol) in DCM (10 ml) was added to a mixture of ethyl 1-(2-chlorophenyl)-4-(hydroxymethyl)-5-(4-hydroxyphenyl)-1H-pyrazole-3-carboxylate (849 mg, crude) and TEA (330 μl, 2.37 mmol) in DCM (40 ml) at −78° C., under N₂ (g). The reaction was continued at −78° C. for 1 hour. Water was added, the phases were separated and the organic phase was evaporated (1.15 g, crude).

MS m/z 533, 535 (M+H)⁺.

### Step H: 1-(2-chlorophenyl)-4-(hydroxymethyl)-5-(4-{[(3,3,3-trifluoropropyl)sulfonyl]oxy}phenyl)-1H-pyrazole-3-carboxylic acid

A solution of LiOH (208 mg, 8.68 mmol) in water (10 ml) was added to a mixture of ethyl 1-(2-chlorophenyl)-4-(hydroxymethyl)-5-(4-{[(3,3,3-trifluoropropyl)sulfonyl]oxy}phenyl)-1H-pyrazole-3-carboxylate (1.15 g, crude) in methanol/THF (1:1, 20 ml) at 0° C. The reaction was continued at 0° C. for 30 minutes. The mixture was made acidic by addition of HCl (1M, aq), and then extracted with ethyl acetate. The organic phase was washed with water and dried over Na₂SO₄ (954 mg, crude).

MS m/z 503, 505 (M−H)⁻.

### Step I: 4-[1-(2-chlorophenyl-3-[(cyclohexylamino)carbonyl]-4-(hydroxymethyl)-1H-pyrazol-5-yl]phenyl 3,3,3-trifluoropropane-1-sulfonate

A solution of cyclohexanamine (68 μl, 0.59 mmol) in DCM (2 ml) was added to a mixture of 1-(2-chlorophenyl)-4-(hydroxylmethyl)-5-(4-{[(3,3,3-trifluoropropyl)sulfonyl]oxy}phenyl)-1H-pyrazole-3-carboxylic acid (300 mg, crude), TEA (83 μl) and benzotriazol-1-yl-oxytri-pyrrolidinophosphonium hexafluorophosphate (312 mg, 0.60 mmol) in DCM (8 ml) at 0° C. The reaction was continued at 0° C. for 15 minutes and then at room temperature for 19 hours. Water was added, the phases separated and the organic phase evaporated. The product was purified further by preparatory HPLC (kromasil C8 column, ammonium acetate (aq, 0.1 M):acetonitrile, product came at 100% acetonitrile) to give an almost white powder (113 mg, 29% yield for 7 steps).

¹H NMR (399.964 MHz) [symbol] 7.44-7.29 (m, 4H), 7.22-7.15 (m, 4H), 7.00 (d, 1H), 5.42-5.30 (br, 1H), 4.60 (d, 2H), 4.01-3.89 (m, 1H), 3.48-3.40 (m, 2H), 2.82-2.68 (m, 2H), 2.06-1.96 (m, 2H), 1.80-1.10 (m, 8H). HRMS Calcd for [C₂₆H₂₇ClF₃N₃O₅S+H]⁺: 586.139. Found: 586.139.

### Example 16

### 4-[1-(2-chlorophenyl)-3-({[(1S,2R)-2-hydroxycyclohexyl]amino}carbonyl)-4-(hydroxymethyl)-1H-pyrazol-5-yl]phenyl 3,3,3-trifluoropropane-1-sulfonate and 4-[1-(2-chlorophenyl)-3-({[(1R,2S)-2-hydroxycyclohexyl]amino}carbonyl)-4-(hydroxymethyl-1H-pyrazol-5-yl]phenyl 3,3,3-trifluoropropane-1-sulfonate

A suspension of cis-2-aminocyclohexanol hydrochloride (97 mg, 0.64 mmol) in DCM (5 ml) was added to a mixture of 1-(2-chlorophenyl)-4-(hydroxymethyl)-5-(4-{[(3,3,3-trifluoropropyl)sulfonyl]oxy}phenyl)-1H-pyrazole-3-carboxylic acid, Ex. 15, Step H (300 mg, crude), TEA (166 μl) and benzotriazol-1-yl-oxytri-pyrrolidinophosphonium hexafluorophosphate (324 mg, 0.62 mmol) in DCM (8 ml) at 0° C. The reaction was continued at 0° C. for 15 minutes and then at room temperature for 20 hours. Water was added, the phases separated and the organic phase evaporated. The product was purified further by preparatory HPLC (kromasil C8 column, ammonium acetate (aq, 0.1 M):acetonitrile, product came at 93% acetonitrile) to give an almost white powder (123 mg, 31% yield for 7 steps).

¹H NMR (399.964 MHz) [symbol] 7.46-7.28 (m, 5H), 7.25-7.15 (m, 4H), 5.25 (t, 1H), 4.60 (d, 2H), 4.15-4.06 (m, 1H), 4.06-4.00 (m, 1H), 3.48-3.40 (m, 2H), 2.82-2.68 (m, 2H), 2.15-1.95 (br, 1H), 1.83-1.34 (m, 8H). HRMS Calcd for [C₂₆H₂₇ClF₃N₃O₆S+H]⁺: 602.134.

Found: 602.135.

1. A compound of formula (I) 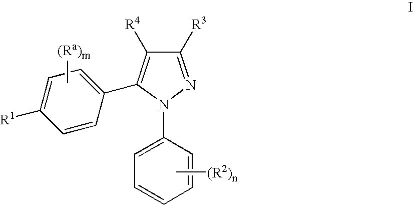
and pharmaceutically acceptable salts thereof, in which
R¹ represents a) a C₁₋₃alkoxy group optionally substituted by one or more of the following
i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group b) R¹ represents a C₄₋₆alkoxy group optionally substituted by one or more of the following i) fluoro ii) a group NR^{c}R^{d} in which R^{c} and R^{d} independently represent H, a C₁₋₆alkyl group or C₁₋₆alkoxycarbonyl group or iii) a 1,3-dioxolan-2-yl group c) a group of formula phenyl(CH₂)ₚO— in which p is 1, 2 or 3 and the phenyl ring is optionally substituted by 1, 2 or 3 groups represented by Z, d) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z e) a group of formula (R⁶)₃ Si in which R⁶ represents a C₁₋₆alkyl group which may be the same or different or f) a group of formula R^{b}O(CO)O in which R^{b} represents a C₁₋₆alkyl group optionally substituted by one or more fluoro;
R^{a} represents halo, a C₁₋₃alkyl group or a C₁₋₃alkoxy group
m is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜHet in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, a C₁₋₆alkoxycarbonyl group or a heterocyclic amine selected from morpholinyl, pyrrolidinyl, piperidinyl or piperazinyl in which the heterocyclic amine is optionally substituted by a C₁₋₃alkyl group or hydroxyl.

2. A compound as claimed in claim 1 in which R³ represents a group as described in paragraph a) above of the R³ definition.

3. A compound as claimed in claim 1 as represented by formula IA 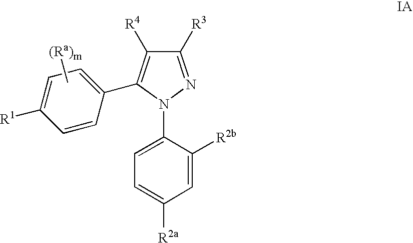
in which R¹ is
a) a C₄₋₆alkoxy group optionally substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z d) a group of formula (R⁶)₃ Si in which R⁶ represents a C₁₋₆alkyl group which may be the same or different or e) a group of formula R^{b}O(CO)O in which R^{b} represents a C₁₋₆alkyl group optionally substituted by one or more fluoro;
R^{a} represents halo and m is 0, 1 or 2;
R^{2a} represents H or chloro;
R^{2b} represents chloro;
R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by a C₁₋₆alkoxycarbonyl group or by one or more fluoro or hydroxy or R⁸ represents pyridyl optionally substituted by one or more W; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

4. A compound as claimed in claim 1 as represented by formula IB 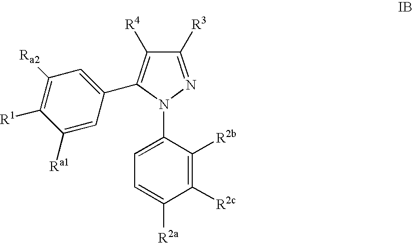
in which R¹ is
a) a C₄₋₆alkoxy group optionally substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;
R^{a1} represents halo or H;
R^{a2} represents halo or H;
R^{2a} represents H or chloro;
R^{2b} represents chloro;
R^{2c} represents halo or H;
R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by one or more fluoro or hydroxy or R⁸ represents pyridyl optionally substituted by trifluoromethyl; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

5. A compound as claimed in claim 1 as represented by formula IC 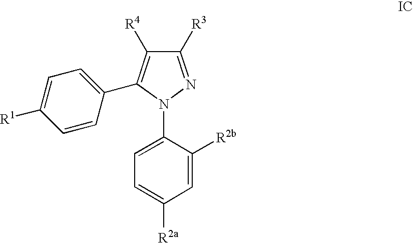
in which R¹ is
a) a C₄₋₆alkoxy group optionally substituted by one or more fluoro, b) a group R⁵S(O)₂O in which R⁵ represents a C₁₋₆alkyl group optionally substituted by one or more fluoro;
R^{2a} represents H or chloro;
R^{2b} represents chloro;
R³ represents a group CONHNR⁷R⁸ in which NR⁷R⁸ represents piperidino or morpholino or R³ represents a group CONHR⁸ in which R⁸ represents a C₅₋₇cycloalkyl group optionally substituted by a C₁₋₆alkoxycarbonyl group or by one or more fluoro or hydroxy or R⁸ represents pyridyl optionally substituted by trifluoromethyl; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆allyl group.

6. A compound as claimed in any previous claim in which R¹ represents a C₄₋₆alkoxy group optionally substituted by one or more fluoro.

7. A compound as claimed in any one of claims 1 to 5 in which R¹ is a C₄₋₆alkoxy group substituted by one or more fluoro.

8. A compound as claimed in any one of claims 1 to 5 in which R¹ represents a group R⁵S(O)₂O in which R⁵ represents a C₁₋₆alkyl group optionally substituted by one or more fluoro.

9. A compound as claimed in any previous claim in which R⁴ represents a group of formula CH₂NR^{e}R^{f} in which R^{e} and R^{f} are as previously defined.

10. A compound as claimed in one of claims 1 to 8 in which R⁴ represents a group of formula CH₂OH.

11. A compound as claimed in any previous claim in which R³ represents N-(piperidin-1-yl)carbamoyl, N-(4,4-difluorocyclohexyl)carbamoyl, N-(5-trifluoromethyl-2-pyridyl)carbamoyl or N-(2-hydroxycyclohexyl)carbamoyl.

12. A compound selected from one or more of the following:
propane-1-sulfonic acid 4-[2-(2,4-dichlorophenyl)-4-hydroxymethyl-5-(piperidin-1-ylcarbamoyl)-2H-pyrazol-3-yl]phenyl ester;
propane-1-sulfonic acid 4-[4-aminomethyl-2-(2,4-dichlorophenyl)-5-(piperidin-1-ylcarbamoyl)-2H-pyrazol-3-yl]phenyl ester;
1-(2,4-dichlorophenyl)-4-hydroxymethyl-5-(4-methoxyphenyl)-1H-pyrazole-3 carboxylic acid (4,4-difluoro-cyclohexyl)amide;
3,3,3-trifluoropropane-1-sulfonic acid 4-[2-(2,4-dichlorophenyl)-5-(4,4-difluoro-cyclohexylcarbamoyl)-4-hydroxymethyl-2H-pyrazol-3-yl]phenyl ester;
3,3,3-trifluoropropane-1-sulfonic acid 4-[2-(2,4-dichlorophenyl)-4-hydroxymethyl-5-(piperidin-1-ylcarbamoyl)-2H-pyrazol-3-yl]phenyl ester;
propane-1-sulfonic acid 4-[2-(2,4-dichlorophenyl)-5-(4,4-difluorocyclohexylcarbamoyl)-4-hydroxymethyl-2H-pyrazol-3-yl]phenyl ester;
3,3,3-trifluoropropane-1-sulfonic acid 4-[2-(2,4-dichlorophenyl)-4-hydroxymethyl-5-(5-trifluoromethylpyridin-2-ylcarbamoyl)-2H-pyrazol-3-yl]phenyl ester;
3,3,3-trifluoropropane-1-sulfonic acid 4-[2-(2,4-dichlorophenyl)-5-(2-hydroxy-cyclohexylcarbamoyl)-4-hydroxymethyl-2H-pyrazol-3-yl]phenyl ester;
3,3,3-trifluoropropane-1-sulfonic acid 4-[5-cyclohexylcarbamoyl-2-(2,4-dichloro-phenyl)-4-hydroxymethyl-2H-pyrazol-3-yl]phenyl ester,
N-cyclohexyl-1-(2,4-dichlorophenyl)-4-hydroxymethyl-5-[4-(3,3,3-trifluoropropoxy)phenyl]-1H-pyrazole-3-carboxamide;
1-(2,4-Dichlorophenyl)-N-(4,4-difluorocyclohexyl)-4-hydroxymethyl-5-[4-(3,3,3-trifluoropropoxy)phenyl]-1H-pyrazole-3-carboxamide;
N-cyclohexyl 1-(2,4-dichlorophenyl)-4-[(dimethylamino)methyl]-5-[4-(3-fluoropropoxy)phenyl]-1H-pyrazole-3-carboxamide;
N-cyclohexyl 1-(2,4-dichlorophenyl)-4-[(dimethylamino)methyl]-5-[4-(3-fluoropropoxy)phenyl]-1H-pyrazole-3-carboxamide;
3,3,3-Trifluoropropane-1-sulfonic acid 4-[5-cyclohexylcarbamoyl-2-(2,4-dichloro-phenyl)-4-dimethylaminomethyl-2H-pyrazol-3-yl]phenyl ester;
4-[1-(2-chlorophenyl)-3-[(cyclohexylamino)carbonyl]-4-(hydroxymethyl)-1H-pyrazol-5-yl]phenyl 3,3,3-trifluoropropane-1-sulfonate;
4-[1-(2-chlorophenyl)-3-({[(1S,2R)-2-hydroxycyclohexyl]amino}carbonyl)-4-(hydroxymethyl)-1H-pyrazol-5-yl]phenyl 3,3,3-trifluoropropane-1-sulfonate and 4-[1-(2-chlorophenyl)-3-({[(1R,2S)-2-hydroxycyclohexyl]amino}carbonyl)-4-(hydroxymethyl)-1H-pyrazol-5-yl]phenyl 3,3,3-trifluoropropane-1-sulfonate.
as well as pharmaceutically acceptable salts thereof.

13. A compound of formula I as claimed in any one of claims 1 to 12 for use as a medicament.

14. A pharmaceutical formulation comprising a compound of formula I as claimed in any one of claims 1 to 12 and a pharmaceutically acceptable adjuvant, diluent or carrier.

15. Use of a compound of formula I as claimed in as claimed in any one of claims 1 to 12 in the preparation of a medicament for the treatment or prophylaxis of obesity, psychiatric disorders such as psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, and neurological disorders such as dementia, neurological disorders, Parkinson's Disease, Huntington's Chorea and Alzheimer's Disease, immune, cardiovascular, reproductive and endocrine disorders, septic shock, diseases related to the respiratory and gastrointestinal systems, and extended abuse, addiction and/or relapse indications.

16. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula I as claimed in as claimed in any one of claims 1 to 12 to a patient in need thereof.

17. A compound as defined in as claimed in any one of claims 1 to 12 for use in the treatment of obesity.

18. A process for the preparation of a compound of formula I as claimed in claim 1 comprising one of the following:
a) reacting a compound of formula II 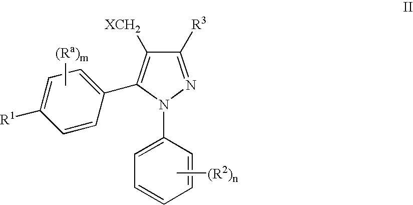
in which R^{a}, R¹, R², R³, m and n are as defined in claim 1 and X represents phthalimido with hydrazine hydrate in the presence of a solvent at a temperature in the range of 15-150° C. to give a compound of formula I in which R^{a}, R¹, R², R³, m and n are as defined in claim 1 and R⁴ represents CH₂NH₂;
b) reacting a compound of formula II 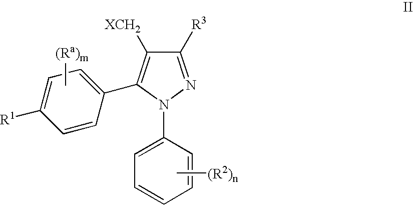
in which R^{a}, R¹, R², R³, m and n are as defined in claim 1 and X represents a leaving group with a hydrolysing agent in the presence of a solvent system at a temperature in the range of 15-150° C. to give a compound of formula I in which R^{a}, R¹, R², R³, m and n are as defined in claim 1 and R⁴ represents CH₂OH;
c) reacting a compound of formula II 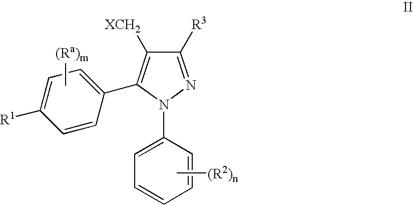
in which R^{a}, R¹, R², R³, m and n are as defined in claim 1 and X represents a leaving group with an amine of formula HNR^{e}R^{f} in which R^{e} and R^{f} are as defined in claim 1 in an inert solvent at a temperature in the range of 15-150° C. to give compounds of formula I in which R^{a}, R¹, R², R³, m and n are as defined in claim 1 and R⁴ represents CH₂NR^{e}R^{f} in which R^{e} and R^{f} are as defined in claim 1;
d) reacting a compound of formula III 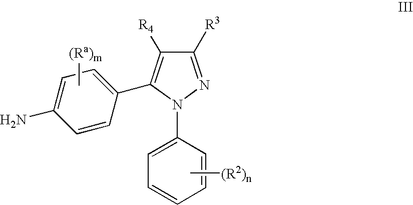
in which R^{a}, R², R³, R⁴, m and n are as defined in claim 1 with a sulphonating agent of formula R⁵SO₂L in which R⁵ is as defined in claim 1 and L represents a leaving group in an inert solvent in the presence of a base at a temperature in the range of −25° C. to 150° C. to give compounds of formula I in which R^{a}, R², R³, R⁴, m and n are as defined in claim 1 and R¹ represents a group R⁵S(O)₂NH;
e) reacting a compound of formula IV 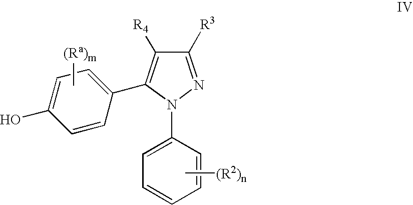
in which R^{a}, R², R³, R⁴, m and n are as defined in claim 1 with either
i) an alkylating agent of formula R⁹X in which R⁹ represents a C₁₋₃alkyl group substituted by one or more fluoro or C₄₋₆alkyl group optionally substituted by one or more fluoro and
X represents a leaving group, in an inert solvent, in the presence of a base, at a temperature in the range of −25° C. to 150° C.; or
ii) an alkylating agent of formula R⁹X in which R⁹ represents a group of formula phenyl(CH₂)ₚ— in which p is 1, 2 or 3 and the phenyl ring is optionally substituted by 1, 2 or 3 groups represented by Z, and X represents a leaving group, in an inert solvent, in the presence of a base, at a temperature in the range of −25° C. to 150° C.; or
iii) a sulphonating agent of formula R⁵SO₂L in which R⁵ is as defined in claim 1 and L represents a leaving group in an inert solvent, in the presence of a base, at a temperature in the range of −25° C. to 150° C.;
respectively, to give compounds of formula I in which R¹ represents i) a C₁₋₃alkoxy group substituted by one or more fluoro or C₄₋₆alkoxy group optionally substituted by one or more fluoro or ii) a group of formula phenyl(CH₂)ₚO— in which p is 1, 2 or 3 and the phenyl ring is optionally substituted by 1, 2 or 3 groups represented by Z, or iii) a group R⁵S(O)₂O, respectively;
f) reacting a compound of formula V 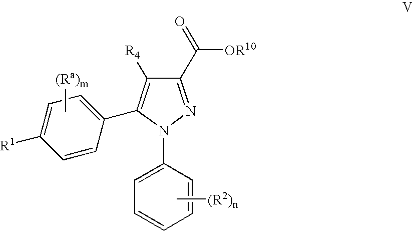
in which R^{a}, R¹, R², R⁴, m and n are as defined in claim 1 and R¹⁰ represents a C₁₋₆alkyl group with a compound of formula VI
R⁷R⁸YNH₂ VI
in which Y, R⁷ and R⁸ are as defined in claim 1 or a salt thereof in an inert solvent, in the presence of a Lewis acid, at a temperature in the range of −25° C. to 150° C. to give compounds of formula I in which R^{a}, R¹, R², R⁴, m and n are as defined in claim 1 and R³ represents a group X—Y—NR⁷R⁸ in which X is CO, Y is absent or represents NH optionally substituted by a C₁₋₃alkyl group and R⁷ and R⁸ are as defined in claim 1 or
g) reacting a compound of formula VII 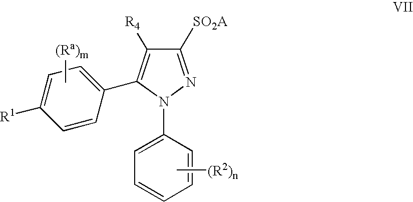
in which R^{a}, R¹, R², R⁴, m and n are as defined in claim 1 and A represents a leaving group, with a compound of formula VI in which Y, R⁷ and R⁸ are as defined in claim 1 or a salt thereof in an inert solvent in the presence of a base, at a temperature in the range of −25° C. to 150° C. to give compounds of formula I in which R³ represents a group X—Y—NR⁷R⁸ in which X is SO₂, Y is absent or represents NH optionally substituted by a C₁₋₃alkyl group and R⁷ and R⁸ are as defined in claim 1.

19. A compound of formula IV 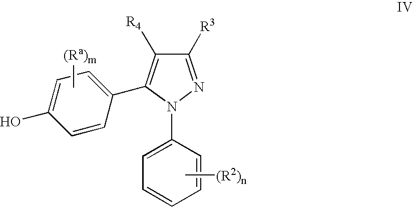
in which R^{a}, R², R³, R⁴, m and n are as defined in claim 1.
